SEQUENCE LISTING (94 sequences)

SEQ ID NO: 1 - protein, 16 aa, Arachis hypogaea
NLRPCEQHLMQKIQRD

SEQ ID NO: 2 - protein, 15 aa, Arachis hypogaea
ERDPYSPSQDPYSPS

SEQ ID NO: 3 - protein, 9 aa, Arachis hypogaea
SDRLQGRQQ

SEQ ID NO: 4 - protein, 9 aa, Arachis hypogaea
RRCQSQLER

SEQ ID NO: 5 - protein, 10 aa, Arachis hypogaea
HASARQQWEL

SEQ ID NO: 6 - protein, 17 aa, Arachis hypogaea
RQQEQQFKRELRNLPQQ

SEQ ID NO: 7 - protein, 7 aa, Arachis hypogaea
PQRCDLE

SEQ ID NO: 8 - protein, 13 aa, Arachis hypogaea
CDLEVESGGRDRY

SEQ ID NO: 9 - protein, 13 aa, Arachis hypogaea
CEALQQIMENQSD

SEQ ID NO: 10 - protein, 12 aa, Arachis hypogaea
CNELNEFENNQR

SEQ ID NO: 11 - protein, 12 aa, Arachis hypogaea
PRPCEQHLMQKI

SEQ ID NO: 12 - protein, 16 aa, Arachis hypogaea
ELQGDRRRCQSQLERA

SEQ ID NO: 13 - protein, 14 aa, Arachis hypogaea
DPYSPSDRRGAGSS

SEQ ID NO: 14 - protein, 14 aa, Arachis hypogaea
MRRERGRGQDSSSS

SEQ ID NO: 15 - protein, 11 aa, Arachis hypogaea
KPCEQHIMQRI

SEQ ID NO: 16 - protein, 7 aa, Arachis hypogaea
YDSYDIR

SEQ ID NO: 17 - protein, 12 aa, Arachis hypogaea
CDELNEMENTQR

SEQ ID NO: 18 - protein, 13 aa, Arachis hypogaea
CEALQQIMENQCD

SEQ ID NO: 19 - protein, 10 aa, Arachis hypogaea
KRELRMLPQQ

SEQ ID NO: 20 - protein, 13 aa, Arachis hypogaea
CNFRAPQRCDLDV

SEQ ID NO: 21 - protein, 19 aa, Arachis hypogaea
GEQEQYDSYNFGSTRSSDQ

SEQ ID NO: 22 - protein, 4 aa, Arachis hypogaea
QDRQ

SEQ ID NO: 23 - protein, 7 aa, Arachis hypogaea
SCERQVD

SEQ ID NO: 24 - protein, 12 aa, Arachis hypogaea
IRSTRSSDQQQR

SEQ ID NO: 25 - protein, 6 aa, Arachis hypogaea
QDRQMV

SEQ ID NO: 26 - protein, 15 aa, Arachis hypogaea
ATHAKSSPYQKKTEN

SEQ ID NO: 27 - protein, 15 aa, Arachis hypogaea
LQSCQQEPDDLKQKA

SEQ ID NO: 28 - protein, 15 aa, Arachis hypogaea
RCTKLEYDPRCVYDP

SEQ ID NO: 29 - protein, 15 aa, Arachis hypogaea
KLEYDPRCVYDPRGH

SEQ ID NO: 30 - protein, 15 aa, Arachis hypogaea
YDPRGHTGTTNQRSP

SEQ ID NO: 31 - protein, 15 aa, Arachis hypogaea
RSPPGERTRGRQPGD

SEQ ID NO: 32 - protein, 15 aa, Arachis hypogaea
PGERTRGRQPGDYDD

SEQ ID NO: 33 - protein, 15 aa, Arachis hypogaea
PGDYDDDRRQPRREE

SEQ ID NO: 34 - protein, 15 aa, Arachis hypogaea
DRRQPRREEGGRWGP

SEQ ID NO: 35 - protein, 15 aa, Arachis hypogaea
AGPREREREEDWRQP

SEQ ID NO: 36 - protein, 15 aa, Arachis hypogaea
REREREEDWRQPRED

SEQ ID NO: 37 - protein, 15 aa, Arachis hypogaea
RQPREDWRRPSHQQP

SEQ ID NO: 38 - protein, 15 aa, Arachis hypogaea
REDWRRPSHQQPRKI

SEQ ID NO: 39 - protein, 15 aa, Arachis hypogaea
PSHQQPRKIRPEGRE

SEQ ID NO: 40 - protein, 15 aa, Arachis hypogaea
RPEGREGEQEWGTPG

SEQ ID NO: 41 - protein, 15 aa, Arachis hypogaea
REETSRNNPFYFPSR

SEQ ID NO: 42 - protein, 15 aa, Arachis hypogaea
NNPFYFPSRRFSTRY

SEQ ID NO: 43 - protein, 15 aa, Arachis hypogaea
SGFISYILNRHDNQN

SEQ ID NO: 44 - protein, 15 aa, Arachis hypogaea
SMPVNTPGQFEDFFP

SEQ ID NO: 45 - protein, 15 aa, Arachis hypogaea
RDQSSYLQGFSRNTL

SEQ ID NO: 46 - protein, 15 aa, Arachis hypogaea
SEEEGDITNPINLRE

SEQ ID NO: 47 - protein, 15 aa, Arachis hypogaea
EGDITNPINLREGEP

SEQ ID NO: 48 - protein, 15 aa, Arachis hypogaea
NNFGKLFEVKPDKKN

SEQ ID NO: 49 - protein, 15 aa, Arachis hypogaea
RYTARLKEGDVFIMP

SEQ ID NO: 50 - protein, 15 aa, Arachis hypogaea
DVFIMPAAHPVAINA

SEQ ID NO: 51 - protein, 15 aa, Arachis hypogaea
PVAINASSELHLLGF

SEQ ID NO: 52 - protein, 15 aa, Arachis hypogaea
LHLLGFGINAENNHR

SEQ ID NO: 53 - protein, 15 aa, Arachis hypogaea
AENNHRIFLAGDKDN

SEQ ID NO: 54 - protein, 15 aa, Arachis hypogaea
NHRIFLAGDKDNVID

SEQ ID NO: 55 - protein, 15 aa, Arachis hypogaea
VIDQIEKQAKDLAFP

SEQ ID NO: 56 - protein, 15 aa, Arachis hypogaea
KQAKDLAFPGSGEQV

SEQ ID NO: 57 - protein, 15 aa, Arachis hypogaea
KDLAFPGSGEQVEKL

SEQ ID NO: 58 - protein, 15 aa, Arachis hypogaea
SHFVSARPQSQSQSP

SEQ ID NO: 59 - protein, 15 aa, Arachis hypogaea
QEEENQGGKGPLLSI

SEQ ID NO: 60 - protein, 15 aa, Arachis hypogaea
AAHASARQQWELQGD

SEQ ID NO: 61 - protein, 15 aa, Arachis hypogaea
WELQGDRRCQSQLER

SEQ ID NO: 62 - protein, 15 aa, Arachis hypogaea
RRCQSQLERANLRPC

SEQ ID NO: 63 - protein, 15 aa, Arachis hypogaea
RPCEQHLMQKIQRDE

SEQ ID NO: 64 - protein, 15 aa, Arachis hypogaea
KIQRDEDSYERDPYS

SEQ ID NO: 65 - protein, 15 aa, Arachis hypogaea
RDEDSYERDPYSPSQ

SEQ ID NO: 66 - protein, 15 aa, Arachis hypogaea
DSYERDPYSPSQDPY

SEQ ID NO: 67 - protein, 15 aa, Arachis hypogaea
PYSPSQDPYSPSPYD

SEQ ID NO: 68 - protein, 15 aa, Arachis hypogaea
CCNELNEFENNQRCM

SEQ ID NO: 69 - protein, 15 aa, Arachis hypogaea
ELNEFENNQRCMCEA

SEQ ID NO: 70 - protein, 15 aa, Arachis hypogaea
LQQIMENQSDRLQGR

SEQ ID NO: 71 - protein, 15 aa, Arachis hypogaea
IMENQSDRLQGRQQE

SEQ ID NO: 72 - protein, 15 aa, Arachis hypogaea
NQSDRLQGRQQEQQF

SEQ ID NO: 73 - protein, 15 aa, Arachis hypogaea
QGRQQEQQFKRELRN

SEQ ID NO: 74 - protein, 15 aa, Arachis hypogaea
KRELRNLPQQCGLRA

SEQ ID NO: 75 - protein, 15 aa, Arachis hypogaea
LPQQCGLRAPQRCDL

SEQ ID NO: 76 - protein, 15 aa, Arachis hypogaea
LRRNALRRPFYSNAP

SEQ ID NO: 77 - protein, 15 aa, Arachis hypogaea
HYEEPHTQGRRSQSQ

SEQ ID NO: 78 - protein, 15 aa, Arachis hypogaea
EPHTQGRRSQSQRPP

SEQ ID NO: 79 - protein, 15 aa, Arachis hypogaea
QGEDQSQQQRDSHQK

SEQ ID NO: 80 - protein, 15 aa, Arachis hypogaea
NTEQEFLRYQQQSRQ

SEQ ID NO: 81 - protein, 15 aa, Arachis hypogaea
PYSPQSQPRQEEREF

SEQ ID NO: 82 - protein, 15 aa, Arachis hypogaea
EGGNIFSGFTPEFLE

SEQ ID NO: 83 - protein, 15 aa, Arachis hypogaea
NIFSGFTPEFLEQAF

SEQ ID NO: 84 - protein, 15 aa, Arachis hypogaea
AIVTVRGGLRILSPD

SEQ ID NO: 85 - protein, 15 aa, Arachis hypogaea
TVRGGLRILSPDRKR

SEQ ID NO: 86 - protein, 15 aa, Arachis hypogaea
EYDEDEYEYDEEDRR

SEQ ID NO: 87 - protein, 15 aa, Arachis hypogaea
YEYDEEDRRRGRGSR

SEQ ID NO: 88 - protein, 15 aa, Arachis hypogaea
IANLAGENSVIDNLP

SEQ ID NO: 89 - protein, 15 aa, Arachis hypogaea
RQLKNNNPFKFFVPP

SEQ ID NO: 90 - protein, 30 aa, Arachis hypogaea
AHASARQQWELQGDRRCQSQLERANLRPCE

SEQ ID NO: 91 - protein, 29 aa, Arachis hypogaea
EDSYERDPYSPSQDPYSPSPYDRRGAGSS

SEQ ID NO: 92 - protein, 30 aa, Arachis hypogaea
QFKRELRNLPQQCGLRAPQRCDLEVESGGR

SEQ ID NO: 93 - protein, 6 aa, Arachis hypogaea
DPYSPS

SEQ ID NO: 94 - protein, 12 aa, Arachis hypogaea
QFKRELRNLPQQ

(19) United States
(12) Patent Application Publication
(10) Pub. No.: US 2026/0077014 A1
(43) Pub. Date: Mar. 19, 2026

(54) COVALENT DUAL-FUNCTIONAL INHIBITORS OF PEANUT ALLERGIES

(71) Applicant: UNIVERSITY OF NOTRE DAME DU LAC, South Bend, IN (US)

(72) Inventors: Zihni Basar BILGICER, Granger, IN (US); Jaeho SHIN, South Bend, IN (US); Emily BROMLEY, South Bend, IN (US); Tanyel KIZILTEPE BILGICER, Granger, IN (US); Jenna Nicole SJOERDSMA, South Bend, IN (US); Gyoyeon HWANG, South Bend, IN (US); Caitlin Lyzette HORGAN, South Bend, IN (US)

(21) Appl. No.: 19/333,170

(22) Filed: Sep. 18, 2025

Related U.S. Application Data
(60) Provisional application No. 63/695,986, filed on Sep. 18, 2024.

(51) Int. Cl. A61K 38/16; A61K 31/137; A61K 39/00; A61K 39/35; A61P 37/08

(52) U.S. Cl. CPC A61K 38/168; A61K 31/137; A61K 39/35; A61P 37/08; A61K 2039/55516; A61K 2039/577

(57) ABSTRACT

The disclosure provides for covalent dual-functional inhibitors of peanut allergic reactions where the inhibitors include a targeting moiety comprising a peanut allergen protein epitope or mimotope peptide sequence, a reactive functional group capable of forming a site-directed covalent bond to the amine moiety of a lysine amino acid of an allergen reactive immunoglobulin, and a variable length spacer comprising oligomers of ethylene glycol monomers, amino acids, saccharides, hydrocarbons, fluorinated hydrocarbons, or a combination thereof, wherein the 10 spacer is conjugated by one or more amide bonds to the TM and the FG.

### RELATED APPLICATIONS

The present application claims priority under 35 U.S.C. § 119 (e) to U.S. Provisional Patent Application No. 63/695,986 filed Sep. 18, 2024, the entire disclosure of which is hereby incorporated by reference in its entirety.

### GOVERNMENT SUPPORT

This invention was made with government support under grant R01 AI108884 awarded by the National Institutes of Health (NIH). The government has certain rights in the invention.

### REFERENCE TO SEQUENCE LISTING

The present application includes a Sequence Listing in electronic format as an xml file titled “501.111US1_SL” which was created on Sep. 17, 2025, and has a size of 82,417 bytes. The contents of xml file 501.111US1_SL are incorporated herein by reference.

### BACKGROUND OF THE INVENTION

The allergic reaction (type I hypersensitivity) is a complex immune reaction to innocuous compounds such as food, environmental factors and drugs. These reactions can cause symptoms from harmless skin irritation to a life-threatening anaphylaxis reaction. The characteristic cellular process of type I hypersensitivity is the release of inflammatory cytokines such as histamine from mast cells and basophils after exposure to an allergen, also known as a degranulation response.

Molecules for the treatment of allergy symptoms include antihistamine drugs such as Diphenhydramine, Cetirizine, and others which are agonists to histamine receptors. Also, there are mast cell stabilizer drugs such as cromolyn which bind calcium channels on mast cells and help to prevent some degranulation events. Finally, there are steroid drugs designed to non-specifically suppress immune system function and therefore allergic reactions. However, these treatments are for post-reaction treatment and/or chronic allergy symptom management and do not prevent degranulation events from occurring.

There have also been some preventative treatments using anti-IgE antibodies, specifically using a monoclonal antibody called Omalizumab to bind to free IgE in patient sera and prevent them from priming mast cells. However, this treatment may be less effective in targeting mast cell bound IgE and may be ineffective in preventing mast cell degranulation. Instead, this treatment is more focused on chronic autoimmune conditions with high levels of IgE in patient sera. Finally, it is also a significantly costly treatment.

The most common form of long-term preventative allergy treatment is immunosuppressive therapy. In this therapy, small doses of antigens are given with adjuvants to modulate the immune system's response to the antigen from IgE mediated to T cell mediated. This has drawbacks in that the treatment takes place over the course of months and cannot be used with highly allergic individuals at the risk of triggering anaphylactic reactions.

There have been attempts to competitively inhibit allergen reactive IgE antibodies by genetic engineering to place a vector coding an allergy reactive peptide into cells and then the cells generate the competitive inhibitor (U.S. Pat. No. 8,349,333 to Wang et al.). This approach appears to be for scientific research purposes and has limited, if any, clinical applications.

To solve the problems described, a substance is needed that can effectively prevent degranulation responses to specific antigens and thus inhibit allergic reactions by covalent inhibition of allergic reactions to foods, and in particular, to peanut allergens. Such inhibitors could be used i) preemptively as a preventative measure by allergy patients who will be in an unknown environment (in situations such as public transportation or air travel, or cating at a restaurant, etc.), ii) as a post-reaction emergency treatment to prevent or stop proliferation of the anaphylactic reaction, or iii) as a treatment to be used in combination with allergy immunotherapy methods. Selective and site-specific covalent antibody modification techniques would be particularly useful both for laboratory use and for potential therapeutics. The present invention satisfies these needs.

### SUMMARY OF THE INVENTION

Allergic reactions are triggered when the patient's immune system reacts to an innocuous substance, as in the case of peanut allergens, through an IgE antibody dependent pathway and result in harmful consequences that in extreme cases may lead to death. We have engineered covalent dual-functional inhibitors (called cMI) to inhibit immune responses to peanut allergens by blocking the responsible immune components specific to peanut allergen recognition without impacting immune defenses. The cMI approach has proven itself as a potent prophylactic agent that inhibits sensitivity to peanut allergens both in vitro tissue culture and in vivo humanized animal models. Furthermore, it has shown promise as a potential treatment to alleviate allergic reaction symptoms if administered immediately after exposure to allergen.

Accordingly, in some embodiments, a cMI peanut allergen antibody inhibitor of Formula I:

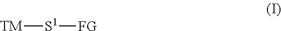

wherein
   TM is a targeting moiety for an antigen binding site (ABS) of an allergen reactive antibody wherein the targeting moiety comprises a peanut allergen protein epitope or mimotope peptide sequence, each having a selective electrostatic affinity for the ABS, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
   FG is a reactive functional group capable of forming a site-directed covalent bond to a lysine amino acid amine moiety of an allergen reactive antibody; and
   S¹ is a variable length spacer comprising oligomers of ethylene glycol, amino acids, saccharides, hydrocarbons, fluorinated hydrocarbons, or a combination thereof, wherein the spacer is conjugated by one or more amide bonds to the TM and the FG;
   wherein when the antibody inhibitor binds to the ABS, and the effective concentration of the FG near the amino acid of the allergen reactive antibody increases to irreversibly inhibit the allergen reactive antibody by the site-directed covalent bond formed by the peanut allergen inhibitor.

In some embodiments, the protein epitope or mimotope peptide sequence of an Ara H1 protein, an Ara H2 protein, an Ara H3 protein, or an Ara H6 protein is any one amino acid sequence according to SEQ ID NO:1 to SEQ ID NO: 93.

In some embodiments, FG comprises a conjugate of an isothiocyanate, an isocyanate, an alkyne, a bromine, an acrylamide, or a maleimide. In some preferred embodiments, the FG is a conjugate of an isothiocyanate or isocyanate. In some preferred embodiments, S¹ comprises an oligomer of y number of ethylene glycol monomers, wherein y is about 2 to about 20.

The disclosure also provides for methods of inhibiting or reducing the severity of an allergic response to a peanut allergy comprising administering an effective amount of an allergen antibody inhibitor according to Formula I to a subject a) prior to exposure of the subject to a peanut allergen, b) after exposure of the subject to a peanut allergen, c) during an allergic response of the subject to a peanut allergen, or d) prior to immunotherapy desensitization of a subject requiring immunotherapy desensitization, wherein inhibition of an antibody to the peanut allergen by the peanut allergen antibody inhibitor inhibits or reduces degranulation of mast cells and basophils, thereby substantially inhibiting or reducing the allergic response of the subject to the peanut allergen;
wherein the peanut allergen antibody inhibitor of Formula I is:

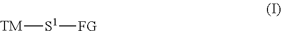

wherein the TM is a targeting moiety for an antigen binding site (ABS) wherein the targeting moiety comprises a peanut allergen protein epitope or mimotope peptide sequence, each having a selective electrostatic affinity for the ABS of an immunoglobulin, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
   FG is a reactive functional group capable of forming a site-directed covalent bond to the amine moiety of a lysine amino acid of an allergen reactive immunoglobulin; and
   S¹ is a variable length spacer comprising an oligomer of monomers of ethylene glycol, amino acids, saccharides, hydrocarbons, fluorinated hydrocarbons, or a combination thereof, wherein the spacer is conjugated by one or more amide bonds to the TM and the FG;
   wherein when the antibody inhibitor binds to the ABS, the effective concentration of the FG near the amino acid of the immunoglobulin increases to irreversibly inhibit the immunoglobulin by the site-directed covalent bond formed by the inhibitor.

In some embodiments, the peanut allergen protein epitope or mimotope may comprise an Ara H1 allergen protein epitope or mimotope comprising an amino acid sequence of any one of SEQ ID NO: 26 to 59.

In some embodiments, the peanut allergen protein epitope or mimotope may comprise an Ara H2 allergen protein epitope or mimotope comprising an amino acid sequence of any one of SEQ ID NO: 1 to SEQ ID NO: 13.

In some embodiments, the peanut allergen protein epitope or mimotope may comprise an Ara H2 allergen protein epitope or mimotope comprising an amino acid sequence of any one of SEQ ID NO: 1 to 13, SEQ ID NO: 60 to 75, and SEQ ID NO: 90 to 92.

In some embodiments, the peanut allergen protein epitope or mimotope may comprise an Ara H3 allergen protein epitope or mimotope comprising an amino acid sequence of any one of SEQ ID NO: 76 to 89.

In some embodiments, the peanut allergen protein epitope or mimotope may comprise an Ara H6 allergen protein epitope or mimotope comprising an amino acid sequence of any one of SEQ ID NO: 14 to SEQ ID NO: 25.

Advantageously, embodiments of the disclosure may be used as a prophylactic to provide protection against peanut allergen for the individuals with the peanut allergy condition. Additionally, it has shown promise as a treatment option, post exposure to allergen, if administered shortly after the exposure. The cMI inhibitors may be used in conjunction with EpiPen immediately following exposure to the peanut allergen. It has shown successful mitigation of allergic reactions.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-E. Peanut Allergen Induced Degranulation. Crude peanut extract (CPE) and Ara h 2 protein were evaluated for their ability to induce an allergic response from RBL-SX38 cells primed with (A)-(B) several mIgE combinations (priming include 38B7 mIgE alone (Column 1), cocktail 1 (Column 2), cocktail 4 (Column 3), cocktail 3 (Column 4), isotype+cocktail 3 (Column 5), or isotype mIgE alone (Column 6)), C) several mIgE combinations or peanut allergic patient samples (for each graph, priming include cocktail 1 (Column 1), cocktail 2 (Column 2), cocktail 3 (Column 3), patient 1 (Column 4), patient 2 (Column 5), or patient 3 (Column 6)), (D)-(E) several mIgE combinations.

FIG. 2A-C. Peanut Allergen Induced Degranulation Inhibition. (A)-(C) Covalent monovalent inhibitors (cMIs) were tested alone and in combination with one another to evaluate their ability to inhibit peanut allergen specific degranulation, clicited by crude peanut extract (CPE) or Ara h 2 protein.

FIG. 3A-B. Kinetic/Mechanistic Experiments Inhibitor Incubation Time Before Washing. (A) Covalent monovalent inhibitors (cMIs) and covalent heterobivalent inhibitors (cHBIs) were tested to evaluate their ability to independently inhibit peanut allergen specific degranulation elicited by crude peanut extract (CPE) in a time dependent manner or (B) in a time dependent manner while in competition with the allergen.

FIG. 4A-V In Vivo Efficacy-Cutaneous and Systemic Anaphylaxis. (A)-(T) Covalent monovalent inhibitors (cMIs) and covalent heterobivalent inhibitors (cHBIs) were tested to evaluate their ability to inhibit peanut allergen specific degranulation in HuNSGS mice, clicited by Ara h 2 protein and compared to a PBS control group. (U)-(V) Skin histology (H&E staining) of various samples at the end of the study. For FIG. 4A, 1=PBS, 2=cHBI (i.v.), 3=cHBI (s.c.), and 4=cMI (i.v.). For FIG. 4B, top graph line is cHBI, middle graph line is cMI, and bottom graph line is PBS. For FIGS. 4F, 4I, 4L, 40 and 4R, square line is PBS, closed circle is cMI iv, and partially closed circle is cMI id.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001, and Hale & Markham, The Harper Collins Dictionary of Biology. Harper Perennial, N.Y. (1991). General laboratory techniques (DNA extraction, RNA extraction, cloning, PCR amplification, cell culturing, etc.) are known in the art and described, for example, in Molecular Cloning: A Laboratory Manual, J. Sambrook et al., 4th edition, Cold Spring Harbor Laboratory Press, 2012; W. C. Chan and P. D. White., “Fmoc Solid Phase Peptide Synthesis: A Practical Approach”, Oxford University Press, Oxford (2004).

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described. The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by us of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

An “effective amount” can also refer to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified.

The term “proximal” refers the distance from the center of the nucleotide binding site (NBS) to the antigen binding site (ABS) or to the lysine moiety which reacts to form a covalent bond with a functional group (FG) on the antibody inhibitor or the antibody ligand. The proximal distance is less than about 50 angstroms (Å), less than about 40 Å, less than about 30 Å, less than about 20 Å, less than about 10 Å, or less than about 5 Å. The proximal distance can also be in a range between about 5 Å to about 50 Å, a range between about 10 Å to about 40 Å, or a range between about 25 Å to about 35 Å.

The term “amino acid” refers to a natural amino acid residue (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acid (e.g. phosphoserine; phosphothreonine; phosphotyrosine; hydroxyproline; gamma-carboxyglutamate; hippuric acid; octahydroindole-2-carboxylic acid; statine; 1,2,3,4,-tetrahydroisoquinoline-3-carboxylic acid; penicillamine; ornithine; citruline; α-methyl-alanine; para-benzoylphenylalanine; phenylglycine; propargylglycine; sarcosine; and tert-butylglycine) residue having one or more open valences. The term also comprises natural and unnatural amino acids bearing amino protecting groups (e.g. acetyl, acyl, trifluoroacetyl, or benzyloxycarbonyl), as well as natural and unnatural amino acids protected at carboxy with protecting groups (e.g. as a (C₁-C₆) alkyl, phenyl or benzyl ester or amide). Other suitable amino and carboxy protecting groups are known to those skilled in the art (see for example, T. W. Greene, Protecting Groups In Organic Synthesis; Wiley: New York, Third Edition, 1999, and references cited therein; D. Voct, Biochemistry, Wiley: New York, 1990; L. Stryer, Biochemistry, (3rd Ed.), W.H. Freeman and Co.: New York, 1975; J. March, Advanced Organic Chemistry, Reactions, Mechanisms and Structure, (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein).

The term “electrostatic affinity” is a synonym for the totality of intermolecular forces that may or may not be present, including but not limited to hydrogen-bonding, Van der Waals forces, London dispersion forces, hydrophobic interactions, and hydrophilic interactions, for molecular binding, i.e., the interaction between two molecules that results in a stable, non-covalent association.

The term “oligomer” means a molecular complex that consists of a few monomer units, for example less than about 20 monomer units, in contrast to a polymer, where the number of monomers is, in principle, not limited.

The term “saccharide” means a sugar such as starch, cellulose, or an oligosaccharide which is a saccharide polymer of about less than about 20 monosaccharides.

The term “epitope” means the part of an antigen molecule to which an antibody attaches itself.

The term “mimotope” means a macromolecule, often a peptide or peptidomimetic, but can also be a small molecule (e.g., a hapten which a small molecule that, when combined with a larger carrier such as a protein, can elicit the production of antibodies that bind specifically to it), which mimics the structure of an epitope. This property causes an antibody response similar to the one elicited by the epitope. As the mimic of binding site, mimotope analysis can be used in mapping epitopes, identifying drug target and inferring protein interaction networks. Furthermore, a mimotope has potential in the development of new diagnostics, therapeutics and vaccines.

The term “conjugate” means joining two or more chemical compounds, for example, by forming a covalent bond between two or more compounds.

The term “avidity” refers to the accumulated strength of multiple affinities of individual non-covalent binding interactions, such as between a protein receptor and its ligand, and is commonly referred to as functional affinity. As such, avidity is distinct from affinity, which describes the strength of a single interaction. However, because individual binding events increase the likelihood of other interactions to occur (i.e. increase the local concentration of each binding partner in proximity to the binding site), avidity is not simply the mere sum of its constituent affinities but is the combined effect of all affinities participating in the biomolecular interaction. Avidity can be applied to antibody interactions in which multiple antigen-binding sites simultaneously interact with the target antigenic epitopes, often in multimerized structures. Individually, each binding interaction may be readily broken; however, when many binding interactions are present at the same time, transient unbinding of a single site does not allow the molecule to diffuse away.

The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CHI, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgGI, lgG2, lgG3, IgG4, IgAQ1 and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CHI, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds HER2 or cathepsin B (also referred to herein as an anti-HER2 antibody or an anti-cathepsin B antibody) will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the terms “peptide,” “polypeptide,” and “protein” arc used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). Furthermore, the algorithm of E. Meyers and W. Miller (Comput. Appl Biosci., 4:11-17 (1988)) incorporated into the ALIGN program (version 2.0) can be used to determine the percent identity between two amino acid sequences, using the PAM120 weight residue table, a Gap length penalty of 12 and a gap penalty of 4. Moreover, the algorithm of Needleman and Wunsch (J Mol Biol. 48:444-453 (1970)) incorporated into the GAP program of the GCG package (available at www.gcg.com) can be used to determine the percent identity between two amino acid sequences, using the Blossum 62 matrix or PAM250 matrix, a gap weight of 16, 14, 12, 10, 8, 6 or 4 and a length weight of 1, 2, 3, 4, 5 or 6.

An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

### Embodiments of the Invention

In various embodiments, the peanut allergen antibody inhibitor or antibody ligand is represented by Formula I:

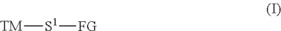

wherein
   TM is a targeting moiety for an antigen binding site (ABS) wherein the targeting moiety comprises a peanut allergen protein epitope or mimotope peptide sequence, each having a selective electrostatic affinity for the ABS of an immunoglobulin, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
   FG is a reactive functional group capable of forming a site-directed covalent bond to the amine moiety of a lysine amino acid of an allergen reactive immunoglobulin; and S¹ is a variable length spacer or linker.

The terms “linker” or “spacer” means a chemical moiety comprising a covalent bond or a chain of atoms that covalently attaches a targeting moiety (TM), a functional group (FG), or a combination thereof, to an antibody inhibitor or antibody ligand. The linker can be an alkyl or aryl chain, including heteroatoms, particularly oxygen or nitrogen, repeating units of ethane, ethylene, ethylene glycol, alkyloxy (e.g. polyethylenoxy, polyethylene glycol (PEG), polymethylencoxy), alkylamino (e.g. polyethylencamino), lipids, saccharides, peptides, amino acids, for example lysine, glycine, alanine, sarcosine, diacid esters and amides, wherein the repeating units can range from 2 to about 20. Furthermore, the repeating units can be linked to the same repeating unit or a different repeating unit by, for example, an amide bond.

Thus, in some embodiments, S¹ comprises an oligomer of ethylene glycol, an oligomer of amino acids, an oligomer of saccharides, one or more hydrocarbons, one or more fluorinated hydrocarbons, or combination thereof, wherein the spacer is conjugated by one or more amide bonds to the TM and the FG. In some preferred embodiments, the S¹ comprises an oligomer of ethylene glycol comprising y number monomers of ethylene glycol. In some embodiments, y is about 2 to about 20. In some embodiments, y is about 4 to about 8, or about 4, or about 8.

In other preferred embodiments, S¹ comprises one or more amino acids, such as, but not limited to, sarcosine, lysine, alanine, and glycine. In other embodiments, S¹ comprises one or more sarcosine residues. In still other embodiments, S¹ may comprise an oligomer of saccharide monomers such as glucose, galactose, fructose, xylose, sucrose, lactose, maltose, isomaltulose, trehalose, sorbitol and mannitol, or an oligosaccharide such as a maltodextrin, raffinose, stachose, or polysaccharides such as a starch (e.g., amylose, amylopectin), glycogen, cellulose, hemicellulose, pectins, and hydrocolloids. Other exemplary linkers that may be used in embodiments of the peanut allergen antibody inhibitor are described, for example, in Handlogten et al., Chemistry & Biology 18, 1179-1188 Sep. 23, 2011.

In some embodiments, the antibody inhibitor binds to the ABS, causing the effective concentration of the FG near the amino acid of the immunoglobulin increases to irreversibly inhibit the immunoglobulin by the site-directed covalent bond formed by the allergen inhibitor.

In some embodiments, the FG comprises a conjugate of an isothiocyanate, an isocyanate, an alkyne, a bromine, an acrylamide, or a maleimide. In some preferred embodiments, the FG is a conjugate of an isothiocyanate or isocyanatc.

In various embodiments of the antibody inhibitors, binding of the TM to the antibody synergistically enhances the avidity of the inhibitor. In other embodiments, the FG is reactive to a lysine residue of an allergen reactive antibody.

In some embodiments, the peanut allergen protein epitope or mimotope comprises an epitope or mimotope of an Ara H1, an Ara H2, an Ara H3, an Ara H6 protein, or an Ara H7 protein.

In some embodiments, the peanut allergen protein epitope or mimotope comprises an epitope or mimotope described in, for example, U.S. Pat. Nos. 10,947,320 and 12,018,092 to Bilgicer et al.; Deak et al, Proc Natl Acad Sci USA. 2019 Apr. 30; 116 (18): 8966-8974; Suarez-Finas et al., Journal of Allergy and Clinical Immunology, Volume 148, Issue 3, 2021, Pages 835-842; and Stanley et al., Archives of Biochemistry and Biophysics, Volume 342, Issue 2, 1997, Pages 244-253.

For example, in some embodiments, the peanut allergen protein epitope or mimotope may comprise an Ara H2 or Ara H6 allergen protein epitope or mimotope comprising one or more amino acid sequence according to: NLRPCEQHLMQKIQRD (SEQ ID NO: 1); ERDPYSPSQDPYSPS (SEQ ID NO: 2); SDRLQGRQQ (SEQ ID NO: 3); RRCQSQLER (SEQ ID NO: 4); HASARQQWEL (SEQ ID NO: 5); RQQEQQFKRELRNLPQQ (SEQ ID NO: 6); PQRCDLE (SEQ ID NO: 7); CDLEVESGGRDRY (SEQ ID NO: 8); CEALQQIMENQSD (SEQ ID NO: 9); CNELNEFENNQR (SEQ ID NO: 10); PRPCEQHLMQKI (SEQ ID NO: 11); ELQGDRRRCQSQLERA (SEQ ID NO: 12); DPYSPSDRRGAGSS (SEQ ID NO: 13); MRRERGRGQDSSSS (SEQ ID NO: 14); KPCEQHIMQRI (SEQ ID NO: 15); YDSYDIR (SEQ ID NO: 16); CDELNEMENTQR (SEQ ID NO: 17); CEALQQIMENQCD (SEQ ID NO: 18); KRELRMLPQQ (SEQ ID NO: 19); CNFRAPQRCDLDV (SEQ ID NO: 20); GEQEQYDSYNFGSTRSSDQ (SEQ ID NO: 21); QDRQ (SEQ ID NO: 22); SCERQVD (SEQ ID NO: 23); IRSTRSSDQQQR (SEQ ID NO: 24); and QDRQMV (SEQ ID NO: 25). In other embodiments, the peanut allergen protein epitope or mimotope comprises at least one of an Ara H1, Ara H2, or Ara H3 protein epitope or mimotope of Table 1:

TABLE 1
Peanut allergen epitope/mimotope library.

| Epitope or | Amino Acid | SEQ ID |
|---|---|---|
| Mimotope | Sequence | NO |
| ara h 1.008 | ATHAKSSPYQKKTEN | 26 |
| ara h 1.015 | LQSCQQEPDDLKQKA | 27 |
| ara h 1.021 | RCTKLEYDPRCVYDP | 28 |
| ara h 1.022 | KLEYDPRCVYDPRGH | 29 |
| ara h 1.025 | YDPRGHTGTTNQRSP | 30 |
| ara h 1.029 | RSPPGERTRGRQPGD | 31 |
| ara h 1.030 | PGERTRGRQPGDYDD | 32 |
| ara h 1.033 | PGDYDDDRRQPRREE | 33 |
| ara h 1.035 | DRRQPRREEGGRWGP | 34 |
| ara h 1.040 | AGPREREREEDWRQP | 35 |
| ara h 1.041 | REREREEDWRQPRED | 36 |
| ara h 1.044 | RQPREDWRRPSHQQP | 37 |
| ara h 1.045 | REDWRRPSHQQPRKI | 38 |
| ara h 1.047 | PSHQQPRKIRPEGRE | 39 |
| ara h 1.050 | RPEGREGEQEWGTPG | 40 |
| ara h 1.056 | REETSRNNPFYFPSR | 41 |
| ara h 1.058 | NNPFYFPSRRFSTRY | 42 |
| ara h 1.090 | SGFISYILNRHDNQN | 43 |
| ara h 1.097 | SMPVNTPGQFEDFFP | 44 |
| ara h 1.103 | RDQSSYLQGFSRNTL | 45 |
| ara h 1.130 | SEEEGDITNPINLRE | 46 |
| ara h 1.131 | EGDITNPINLREGEP | 47 |
| ara h 1.137 | NNFGKLFEVKPDKKN | 48 |
| ara h 1.167 | RYTARLKEGDVFIMP | 49 |
| ara h 1.170 | DVFIMPAAHPVAINA | 50 |
| ara h 1.173 | PVAINASSELHLLGF | 51 |
| ara h 1.176 | LHLLGFGINAENNHR | 52 |
| ara h 1.179 | AENNHRIFLAGDKDN | 53 |
| ara h 1.180 | NHRIFLAGDKDNVID | 54 |
| ara h 1.184 | VIDQIEKQAKDLAFP | 55 |
| ara h 1.186 | KQAKDLAFPGSGEQV | 56 |
| ara h 1.187 | KDLAFPGSGEQVEKL | 57 |
| ara h 1.194 | SHFVSARPQSQSQSP | 58 |
| ara h 1.203 | QEEENQGGKGPLLSI | 59 |
| ara h 2.005 | AAHASARQQWELQGD | 60 |
| ara h 2.008 | WELQGDRRCQSQLER | 61 |
| ara h 2.010 | RRCQSQLERANLRPC | 62 |
| ara h 2.014 | RPCEQHLMQKIQRDE | 63 |
| ara h 2.017 | KIQRDEDSYERDPYS | 64 |
| ara h 2.018 | RDEDSYERDPYSPSQ | 65 |
| ara h 2.019 | DSYERDPYSPSQDPY | 66 |
| ara h 2.021 | PYSPSQDPYSPSPYD | 67 |
| ara h 2.030 | CCNELNEFENNQRCM | 68 |
| ara h 2.031 | ELNEFENNQRCMCEA | 69 |
| ara h 2.036 | LQQIMENQSDRLQGR | 70 |
| ara h 2.037 | IMENQSDRLQGRQQE | 71 |
| ara h 2.038 | NQSDRLQGRQQEQQF | 72 |
| ara h 2.040 | QGRQQEQQFKRELRN | 73 |
| ara h 2.043 | KRELRNLPQQCGLRA | 74 |
| ara h 2.045 | LPQQCGLRAPQRCDL | 75 |
| ara h 3.018 | LRRNALRRPFYSNAP | 76 |
| ara h 3.030 | HYEEPHTQGRRSQSQ | 77 |
| ara h 3.031 | EPHTQGRRSQSQRPP | 78 |
| ara h 3.037 | QGEDQSQQQRDSHQK | 79 |
| ara h 3.060 | NTEQEFLRYQQQSRQ | 80 |
| ara h 3.068 | PYSPQSQPRQEEREF | 81 |
| ara h 3.079 | EGGNIFSGFTPEFLE | 82 |
| ara h 3.080 | NIFSGFTPEFLEQAF | 83 |
| ara h 3.092 | AIVTVRGGLRILSPD | 84 |
| ara h 3.093 | TVRGGLRILSPDRKR | 85 |
| ara h 3.100 | EYDEDEYEYDEEDRR | 86 |
| ara h 3.102 | YEYDEEDRRRGRGSR | 87 |
| ara h 3.152 | IANLAGENSVIDNLP | 88 |
| ara h 3.162 | RQLKNNNPFKFFVPP | 89 |

In some embodiments, the Ara H6 allergen protein epitope or mimotope comprises an amino acid sequence of one or more of MRRERGRGQDSSSS (SEQ ID NO: 14), KPCEQHIMQRI (SEQ ID NO: 15), GEQEQYDSYNFGSTRSSDQ (SEQ ID NO: 21), QDRQ (SEQ ID NO: 22), and SCERQVD (SEQ ID NO: 23).

In some embodiments, the Ara H2 allergen protein epitope or mimotope comprises an amino acid sequence of one or more of:

Ara h 2-Epitope 1:
(SEQ ID NO: 90)
AHASARQQWELQGDRRCQSQLERANLRPCE;
Ara h 2-Epitope 2:
(SEQ ID NO: 91)
EDSYERDPYSPSQDPYSPSPYDRRGAGSS;
Ara h 2-Epitope 3:
(SEQ ID NO: 92)
QFKRELRNLPQQCGLRAPQRCDLEVESGGR.

In some embodiments, the Ara H2 allergen protein epitope or mimotope comprises a portion or fragment of any one of SEQ ID NO: 90 to SEQ ID NO: 92. For example, the portion or fragment may comprise any 3-15 contiguous amino acids from SEQ ID NO: 90, SEQ ID NO: 91, or SEQ ID NO: 92, or about 5-15, about 8-15, or about 8-12 contiguous amino acids from SEQ ID NO: 90, SEQ ID NO: 91, or SEQ ID NO: 92. The efficacy of the portion or fragment may comprise any 3-15 contiguous amino acids from SEQ ID NO: 90, SEQ ID NO: 91, or SEQ ID NO: 92 may be determined using, for example, a Rat Basophil Leukemia cells (RBL) degranulation inhibition assay or similar assays know in the art. In some embodiments, the Ara H2 allergen protein epitope or mimotope comprises HASARQQWEL (SEQ ID NO: 5), DPYSPS (SEQ ID NO: 93), or QFKRELRNLPQQ (SEQ ID NO: 94).

In some embodiments, the Ara H1 protein epitope or mimotope comprises at least one of SEQ ID NO: 31, SEQ ID NO: 32, and SEQ ID NO: 34 to SEQ ID NO: 41. In some embodiments, the Ara H2 protein epitope or mimotope comprises at least one of SEQ ID NO: 78, SEQ ID NO: 83, SEQ ID NO: 86, and SEQ ID No: 87.

In some embodiments, the proline (P) at position 4 of SEQ ID NO: 13; at positions 4, 8, and 11 of SEQ ID NO: 67; and/or at positions 3 and 5 of SEQ ID NO: 93 may be hydroxylated. In some embodiments, the proline (P) at position 4 of SEQ ID NO: 13 is hydroxylated. In some embodiments, the proline (P) at positions 4, 8, and 11 of SEQ ID NO: 67 are hydroxylated. In some embodiments, the proline (P) at positions 3 and 5 of SEQ ID NO: 93 are hydroxylated.

In some embodiments, the peanut allergen is derived from Arachis Sp. In some embodiments, the peanut allergen is derived from Arachis hypogaea.

In some embodiments, the C-terminus of the peanut allergen protein epitope or mimotope peptide sequence is an amide or carboxylic acid.

In some embodiments, a peanut allergen antibody inhibitor of Formula I:

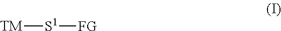

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising and amino acid sequence of any one of SEQ ID NO:1 to SEQ ID NO: 25;
   FG is a reactive functional group capable of forming a site-directed covalent bond to an amine moiety of a lysine amino acid of the allergen reactive antibody; and
   S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally
   wherein when the peanut allergen antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, a peanut allergen antibody inhibitor of Formula I:

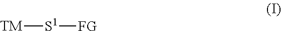

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising an amino acid sequence of any one of SEQ ID NO: 26 to SEQ ID NO: 89;
   FG is a reactive functional group capable of forming a site-directed covalent bond to an amine moiety of a lysine amino acid of the allergen reactive antibody; and
   S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally
   wherein when the peanut allergen antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, a peanut allergen antibody inhibitor of Formula I:

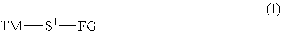

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising and amino acid sequence of any one of SEQ ID NO: 26 to SEQ ID NO: 89;
   FG is a reactive functional group capable of forming a site-directed covalent bond to an amine moiety of a lysine amino acid of the allergen reactive antibody; and
   S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally
   wherein when the peanut allergen antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, a peanut allergen antibody inhibitor of Formula I:

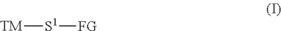

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising between about 3 to about 15 contiguous amino acids of SEQ ID NO: 90, SEQ ID NO: 91, or SEQ ID NO: 92;
   FG is a reactive functional group capable of forming a site-directed covalent bond to an amine moiety of a lysine amino acid of the allergen reactive antibody; and
   S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally
   wherein when the peanut allergen antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, a peanut allergen antibody inhibitor of Formula I:

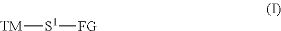

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising and amino acid sequence of any one of SEQ ID NO: 13, SEQ ID NO: 22, SEQ ID NO: 61, SEQ ID NO: 67, SEQ ID NO: 75, and SEQ ID NO: 93;
   FG is a reactive functional group capable of forming a site-directed covalent bond to an amine moiety of a lysine amino acid of the allergen reactive antibody; and
   S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally
   wherein when the peanut allergen antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, a peanut allergen antibody inhibitor of Formula I:

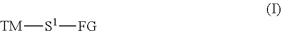

wherein
   TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide comprising and amino acid sequence of any one of SEQ ID NO: 13, SEQ ID NO: 22, SEQ ID NO: 61, SEQ ID NO: 67, SEQ ID NO: 75, and SEQ ID NO: 93;
   FG is a reactive functional group capable of forming a site-directed covalent bond to an amine moiety of a lysine amino acid of the allergen reactive antibody; and
   S¹ is a variable length spacer comprising oligomers of ethylene glycol, wherein a number of the oligomers of ethylene glycol is about 4 to about 8, wherein the spacer is conjugated by one or more amide bonds to the TM and the FG; and optionally
   wherein when the peanut allergen antibody inhibitor binds to the ABS, and the effective concentration of the FG near the lysine amino acid of the allergen reactive antibody increases to irreversibly inhibit the allergen reactive antibody by the site-directed covalent bond formed by the peanut allergen antibody inhibitor.

In some embodiments, the peanut allergen antibody inhibitor may be formulated as a composition comprising one or more peanut allergen antibody inhibitors or a pharmaceutically acceptable salt thereof, and a pharmaceutically acceptable excipient, carrier, or diluent.

In other embodiments, either the peanut allergen antibody inhibitor or a composition thereof may be administered to a person in need thereof. For example, in some embodiments, a method of inhibiting or reducing the severity of an allergic response to a peanut allergy comprising administering an effective amount of a peanut allergen antibody inhibitor according to Formula I to a subject a) prior to exposure of the subject to an allergen, b) after exposure of the subject to a peanut allergen, c) during an allergic response of the subject to a peanut allergen, or d) prior to immunotherapy desensitization of a subject requiring immunotherapy desensitization, wherein inhibition of an immunoglobulin antibody to the peanut allergen prevents degranulation of mast cells and basophils, thereby substantially inhibiting or reducing the allergic response of the subject to the peanut allergen;
wherein the peanut allergen antibody inhibitor of Formula I is:

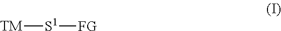

wherein
   TM is a targeting moiety for an antigen binding site (ABS) wherein the targeting moiety comprises a peanut allergen protein epitope or mimotope peptide sequence, each having a selective electrostatic affinity for the ABS of an immunoglobulin, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
   FG is a reactive functional group capable of forming a site-directed covalent bond to the amine moiety of a lysine amino acid of an allergen reactive immunoglobulin; and
   S¹ is a variable length spacer comprising oligomers of ethylene glycol, one or more amino acids, one or more saccharides, one or more hydrocarbons, one or more fluorinated hydrocarbons, or combination thereof, wherein the spacer is conjugated by one or more amide bonds to the TM and the FG; and
   wherein when the antibody inhibitor binds to the ABS, the effective concentration of the FG near the amino acid of the immunoglobulin increases to irreversibly inhibit the immunoglobulin by the site-directed covalent bond formed by the inhibitor.

In some embodiments, the protein epitope or mimotope peptide sequence of an Ara H1 protein, an Ara H2 protein, an Ara H3 protein, or an Ara H6 protein comprises any one of SEQ ID NO: 1 to SEQ ID NO: 94, or a portion thereof (e.g., a portion any one of SEQ ID Nos. 90, 91, 92, etc.).

In some embodiments, the peanut allergen antibody inhibitor is one or more of:

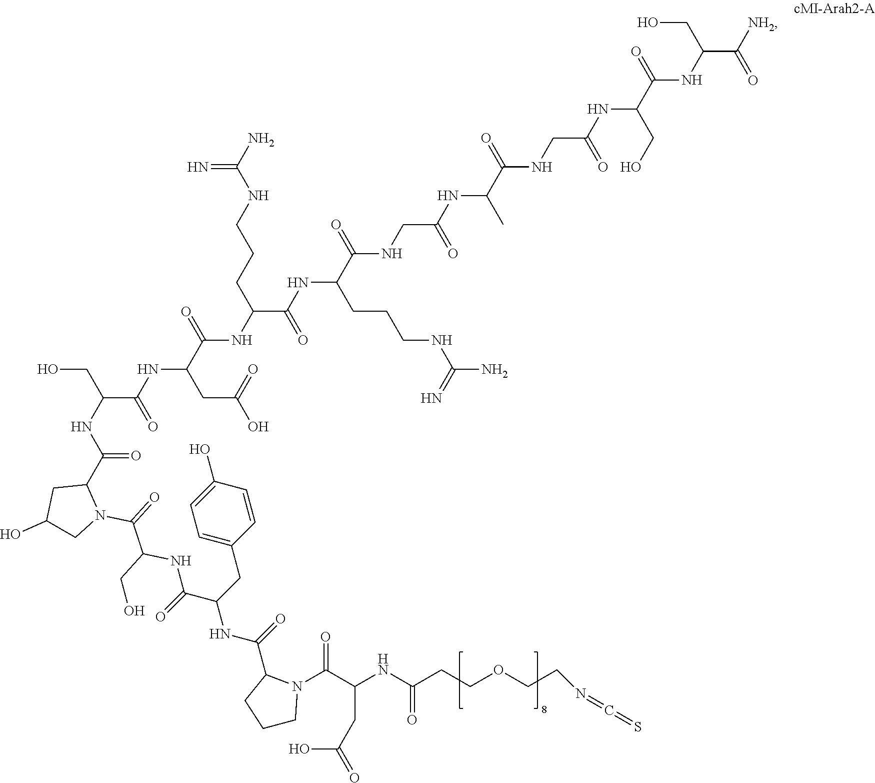

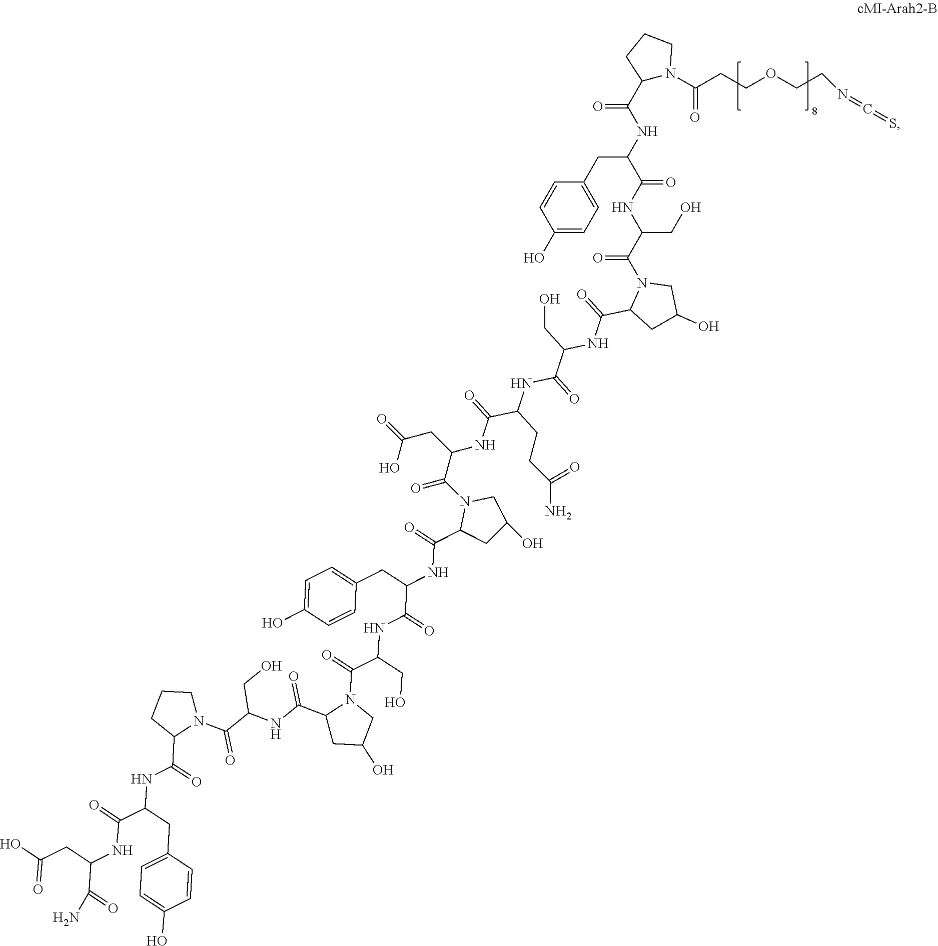

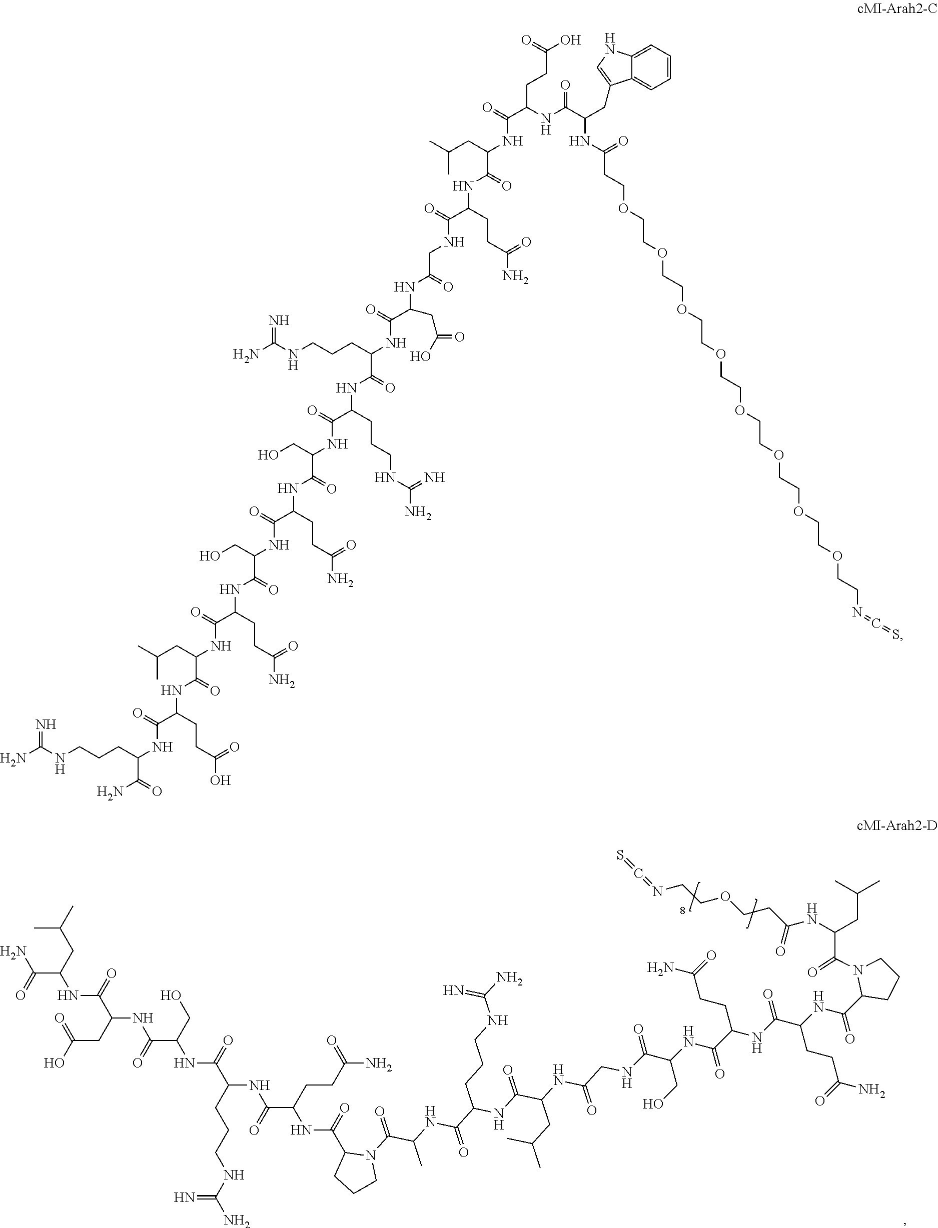

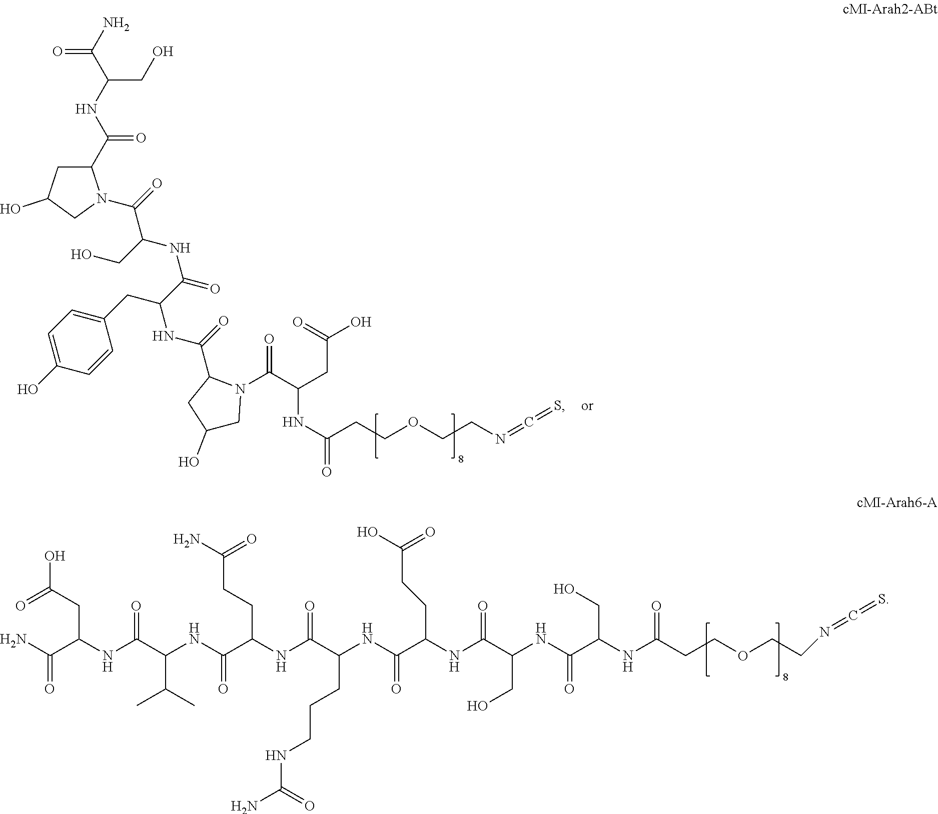

In some embodiments, two or more of the described peanut allergen antibody inhibitors may be administered to a subject in need thereof concurrently or sequentially. In some embodiments, two or more of the described peanut allergen antibody inhibitors may be administered to a subject in need thereof concurrently as a single composition or sequentially as separate compositions. In some embodiments, the same or different peanut allergen antibody inhibitors may be administered concurrently or sequentially to a subject.

In some embodiments, the peanut allergen antibody inhibitor may be co-administered with epinephrine to mitigate an anaphylaxis response in a subject exposed to an allergen.

In some embodiments, an effective amount of the peanut allergen antibody inhibitor may be about 0.1 mg to about 2000 mg, about 0.5 mg to about 1800 mg, about 5 mg to about 1600 mg, about 10 mg to about 1500 mg, about 15 mg to about 1250 mg, or about 20 mg to about 1000 mg. In some embodiments, an effective amount of the peanut allergen antibody inhibitor may be about 10 mg to about 2000 mg, about 100 mg to about 2000 mg, about 110 mg to about 1800 mg, or about 120 mg to about 1600 mg. In some embodiments, an effective amount of the peanut allergen antibody inhibitor may be about 120 mg to about 1200 mg or about 160 mg to about 1600 mg, or about 100 mg to about 800 mg, about 120 to about 650 mg, or about 10 mg to about 50 mg. In other embodiments, the effective amount is about 1000 mg to about 10,000 mg, about 2000 mg to about 8000 mg, about 3000 mg to about 6000 mg, or about 1000 mg, about 1500 mg, about 2000 mg, about 2500 mg, about 3000 mg, about 3500 mg, about 4000 mg, about 4500 mg, about 5000 mg, about 5500 mg, about 6000 mg, about 6500 mg, about 7000 mg, about 7500 mg, about 8000 mg, about 8500 mg, about 9000 mg, about 9500 mg, or about 10000 mg,

In some embodiments, an effective amount of the peanut allergen antibody inhibitor may be about 0.1 mg/kg to about 100 mg/kg, about 0.1 mg/kg to about 75 mg/kg, about 0.1 mg/kg to about 50 mg/kg, about 1 mg/kg to about 40 mg/kg, about 2 mg/kg to about 30 mg/kg, or about 5 mg/kg to about 20 mg/kg. In some embodiments, an effective amount of the peanut allergen antibody inhibitor may be about 1 mg/kg to about 25 mg/kg, about 2 mg/kg to about 20 mg/kg, or about 5 mg/kg to about 15 mg/kg, or about 2 mg/kg, about 4 mg/kg, about 6 mg/kg, about 8 mg/kg, about 10 mg/kg, about 12 mg/kg, about 14 mg/kg, about 16 mg/kg, about 18 mg/kg, about 20 mg/kg, about 22 mg/kg, or about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg.

In some embodiments, the peanut allergen antibody inhibitor may be in a concentration of about 0.1 mg/mL to about 15 mg/mL, about 0.25 mg/mL to about 12 mg/mL, about 0.5 mg/mL to about 10 mg/mL, or about 0.5 to about 8 mg/mL. In some embodiments, the peanut allergen antibody inhibitor may be in a concentration of about 0.6 mg/mL, about 2 mg/mL, about 3 mg/mL, about 7 mg/mL, or about 10 mg/mL.

In some embodiments, the inhibitor is administered to a subject to achieve an average peanut allergen antibody inhibitor concentration of about 0.1 μm to 5 μm, or about 0.5 μm to about 3 μm. In some embodiments, the inhibitor is administered a subject to achieve an average peanut allergen antibody inhibitor concentration of about 0.1 μm to 2.5 μm, or about 1 μm.

In some embodiments, the inhibitor is administered to a subject in an amount of 0.1 nmol to about 250 nmol, about 0.5 nmol to about 225 nmol, about 1 nmol to about 200 nmol, about 5 nmol to about 150 nmol, about 10 nmol to about 100 nmol, or about 20 nmol to about 80 nmol.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and b-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

In some embodiments, the peanut allergen inhibitors or pharmaceutical salts thereof are formulated for oral delivery to a subject, such as through a pill comprising a peanut allergen inhibitor or pharmaceutical salt thereof according to formula I and as described herein, and a pharmaceutically acceptable excipient, carrier, or diluent.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's dict. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations. Methods for converting dosages between humans and other animals are described, for example, in Nair et al., J Basic Clin Pharma 2016; 7:27-31.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. Additional routes and formulations for administration to a subject are disclosed, for example, in U.S. Pat. No. 12,257,229 to Nestor et al.

Compositions may be administered individually to a patient or may be administered in combination (e.g. simultaneously, sequentially, or separately) with other agents, drugs or hormones.

In some preferred embodiments, pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Preferred forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the inhibitor may be in dry form, for reconstitution before use with an appropriate sterile liquid.

Once formulated, the compositions of the invention can be administered directly to the subject. The subjects to be treated can be animals. However, it is preferred that the compositions are adapted for administration to human subjects.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see U.S. Patent Publication No. 2006/0269593 to Glenn et al.), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously (in particular), intraperitoneally, intravenously or intramuscularly, or delivered to the interstitial space of a tissue. The compositions can also be administered into a lesion. Dosage treatment may be a single dose schedule or a multiple dose schedule.

In other embodiments, the inhibitors and compositions comprising the peanut allergen antibody inhibitors may be formulated for use with a transdermal patch. For example, transdermal patches may comprise one or more layers including a liner and/or adhesive tape layer, a membrane layer, a drug formulation layer, and a backing film layer. In other embodiments, transdermal patches may include one or more of a release liner that protects the patch during storage and removed prior to use, a drug composition in direct contact with the release liner, an adhesive layer to adhere the components of the patch together along with adhering the patch to the skin, a membrane layer that controls the release of the drug or drug composition, a backing that protects the patch from the outer environment, optionally, a penetration enhancer and a matrix filler that provides bulk to the matrix in one of the layers, and may act as matrix stiffening agents. Other examples of transdermal patches are described, for example, in U.S. Pat. No. 5,876,746 to Jona et al.; and 5,252,334 to Chiang et al. Also see, for example, Nachum et al. Clin Pharmacokinet 45, 543-566 (2006); Jamaledin et al., J Clin Med. 2020 Feb. 17; 9 (2): 542; Carrithers et al., Patient Prefer Adherence. 2020 Aug. 25; 14:1541-1551; Li et al., Adv Funct Mater. 2021 Oct. 26; 31 (44): 2103359).

### Statements of Certain Embodiments of the Invention

1. In a first embodiment, a peanut allergen antibody inhibitor of Formula I: TM-S¹-FG (I), wherein TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide sequence of an Ara H1 protein, an Ara H2 protein, an Ara H3 protein, or an Ara H6 protein; FG is a reactive functional group capable of forming a site-directed covalent bond to an amine moiety of a lysine amino acid of the allergen reactive antibody; and S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and optionally, wherein when the peanut allergen antibody inhibitor binds to the ABS, and the effective concentration of the FG near the lysine amino acid of the allergen reactive antibody increases to irreversibly inhibit the allergen reactive antibody by the site-directed covalent bond formed by the peanut allergen antibody inhibitor.
2. The inhibitor of embodiment 1, wherein the FG comprises a conjugate of an isothiocyanate, an isocyanate, an alkyne, a bromine, an acrylamide, or a maleimide.
3. The inhibitor of embodiment 1 or 2, wherein the FG is a conjugate of an isothiocyanate or isocyanate.
4. The inhibitor any one of embodiment 1-3, wherein the FG is a conjugate of an isothiocyanate.
5. The inhibitor any one of embodiment 1-4, wherein the FG is isocyanate.
6. The inhibitor of any one of embodiment 1-5, wherein S¹ comprises an oligomer of ethylene glycol monomers comprising y number of monomers of ethylene glycol.
7. The inhibitor of any one of embodiment 1-6, wherein S¹ comprises an oligomer of amino acid comprising y number of amino acids.
8. The inhibitor of any one of embodiment 1-7, wherein S¹ comprises an oligomer of saccharide monomers comprising y number of saccharide monomers.
9. The inhibitor of any one of embodiment 1-8, y is about 1 to about 15.
10. The inhibitor of any one of embodiment 1-9, y is about 3 to about 15.
11. The inhibitor of any one of embodiment 1-10, y oligomers is about 5 to about 15.
12. The inhibitor of any one of embodiment 1-11, y is about 10 to about 15.
13. The inhibitor of any one of embodiment 1-12, y is about 1 to about 5.
14. The inhibitor of any one of embodiments 1-13, wherein S¹ comprises an oligomer of ethylene glycol monomers comprising y number of monomers of ethylene glycol, wherein y is about 2 to about 20.
15. The inhibitor of any one of embodiments 1-14, wherein the y number of monomers of ethylene glycol is about 2 to about 15.
16. The inhibitor of any one of embodiments 1-15, wherein the y number of monomers of ethylene glycol is about 5 to about 10.
17. The inhibitor of any one of embodiments 1-16, wherein the y number of monomers of ethylene glycol is about 8.
18. The inhibitor of any one of embodiments 1-17, wherein the peanut allergen protein epitope or mimotope comprises any one of SEQ ID NO:1 to SEQ ID NO: 25.
19. The inhibitor of any one of embodiments 1-18, wherein the peanut allergen protein epitope or mimotope comprises any one of SEQ ID NO: 26 to SEQ ID NO: 89.
20. The inhibitor of any one of embodiments 1-19, wherein the peanut allergen protein epitope or mimotope comprises one of: (SEQ ID NO: 90), (SEQ ID NO: 91), (SEQ ID NO: 92), and a portion thereof, wherein the portion thereof comprises about 3 to about 15 contiguous amino acids of SEQ ID NO: 90, SEQ ID NO: 91, or SEQ ID NO: 92.
21. The inhibitor of any one of embodiments 1-20, wherein the peanut allergen protein epitope or mimotope comprises one of: (SEQ ID NO: 90), (SEQ ID NO: 91), (SEQ ID NO: 92), and a portion thereof, wherein the portion thereof comprises about 5 to about 15 contiguous amino acids of SEQ ID NO: 90, SEQ ID NO: 91, or SEQ ID NO: 92.
22. The inhibitor of any one of embodiments 1-21, wherein the peanut allergen protein epitope or mimotope comprises one of: (SEQ ID NO: 90), (SEQ ID NO: 91), (SEQ ID NO: 92), and a portion thereof, wherein the portion thereof comprises about 8 to about 15 contiguous amino acids of SEQ ID NO: 90, SEQ ID NO: 91, or SEQ ID NO: 92.
23. The inhibitor of any one of embodiments 1-22, wherein the peanut allergen protein epitope or mimotope comprises one of: (SEQ ID NO: 90), (SEQ ID NO: 91), (SEQ ID NO: 92), and a portion thereof, wherein the portion thereof comprises about 10 to about 15 contiguous amino acids of SEQ ID NO: 90, SEQ ID NO: 91, or SEQ ID NO: 92.
24. The inhibitor of any one of embodiments 1-23, wherein the peanut allergen protein epitope or mimotope comprises SEQ ID NO: 5, SEQ ID NO: 93, or SEQ ID NO: 94.
25. The inhibitor of any one of embodiments 1-24, wherein the peanut allergen protein epitope or mimotope peptide sequence comprises any one of SEQ ID NO: 13, SEQ ID NO: 22, SEQ ID NO: 61, SEQ ID NO: 67, SEQ ID NO: 75, and SEQ ID NO: 93.
26. The inhibitor of any one of embodiments 1-25, wherein the peanut allergen protein epitope or mimotope peptide sequence comprises SEQ ID NO: 13.
27. The inhibitor of any one of embodiments 1-26, wherein the peanut allergen protein epitope or mimotope peptide sequence comprises SEQ ID NO: 22.
28. The inhibitor of any one of embodiments 1-27, wherein the peanut allergen protein epitope or mimotope peptide sequence comprises SEQ ID NO: 61.
29. The inhibitor of any one of embodiments 1-28, wherein the peanut allergen protein epitope or mimotope peptide sequence comprises SEQ ID NO: 67.
30. The inhibitor of any one of embodiments 1-29, wherein the peanut allergen protein epitope or mimotope peptide sequence comprises SEQ ID NO: 75.
31. The inhibitor of any one of embodiments 1-30, wherein the peanut allergen protein epitope or mimotope peptide sequence comprises SEQ ID NO: 93.
32. The inhibitor of any one of embodiments 1-31, wherein the inhibitor comprises any one of structures cMI-Arah2-A, cMI-Arah2-B, cMI-Arah2-C, cMI-Arah2-D, cMI-Arah2-ABt, or cMI-Arah6-A.
33. The inhibitor of any one of embodiments 1-32, wherein the peanut allergen protein epitope or mimotope comprises an Ara H1 allergen protein epitope or mimotope comprising an amino acid sequence of any one of: SEQ ID NO: 26 to 59.
34. The inhibitor of any one of embodiments 1-33, wherein peanut allergen protein epitope or mimotope comprises an Ara H2 allergen protein epitope or mimotope comprising an amino acid sequence of any one of SEQ ID NO: 1 to SEQ ID NO: 13.
35. The inhibitor of any one of embodiments 1-34, wherein the peanut allergen protein epitope or mimotope comprises an Ara H2 allergen protein epitope or mimotope comprising an amino acid sequence of any one of SEQ ID NO: 1 to13, SEQ ID NO: 60 to 75, and SEQ ID NO: 90 to 92.
36. The inhibitor of any one of embodiments 1-35, wherein the peanut allergen protein epitope or mimotope comprises an Ara H3 allergen protein epitope or mimotope comprising an amino acid sequence of any one of SEQ ID NO: 76 to 89.
37. The inhibitor of any one of embodiments 1-36, wherein the peanut allergen protein epitope or mimotope comprises an Ara H6 allergen protein epitope or mimotope comprising an amino acid sequence of any one of SEQ ID NO: 14 to SEQ ID NO: 25.
38. The inhibitor of any one of embodiments 1-36, wherein the proline (P) at position 4 of SEQ ID NO: 13 is hydroxylated.
39. The inhibitor of any one of embodiments 1-37, wherein the proline (P) at positions 4, 8, and 11 of SEQ ID NO: 67 are hydroxylated
40. The inhibitor of any one of embodiments 1-38, wherein the proline (P) at positions 3 and 5 of SEQ ID NO: 93 are hydroxylated
41. A composition comprising the inhibitor of any of embodiments 1-40, and a pharmaceutically acceptable excipient, carrier, or diluent.
42. The composition of embodiment 41, wherein the inhibitor of any one of embodiments 1-41 are present in the composition in an amount of about 0.1 mg to about 10,000 mg or 0.1 mg to about 5,000 mg.
43. The composition of embodiment 41 or 42, wherein the inhibitor of any one of embodiments, 1-40 are present in the composition in an amount of about 1 mg to about 2500 mg.
44. The composition of any one of embodiment 41-43, wherein the, an effective amount of the peanut allergen antibody inhibitor may be a dosage of about 0.1 mg/kg to about 50 mg/kg.
45. The composition of any one of embodiment 41-44, wherein the, an effective amount of the peanut allergen antibody inhibitor may be a dosage of about 0.1 mg/kg to about 25 mg/kg.
46. A method of inhibiting or reducing the severity of an allergic response to a peanut allergy comprising administering an effective amount of a peanut allergen antibody inhibitor according to Formula I to a subject a) prior to exposure of the subject to an allergen, b) after exposure of the subject to an allergen, c) during an allergic response of the subject to an allergen, or d) prior to immunotherapy desensitization of a subject requiring immunotherapy desensitization, wherein inhibition of an immunoglobulin antibody to the allergen by the peanut allergen antibody inhibitor inhibits or reduces degranulation of mast cells and basophils, thereby substantially inhibiting or reducing the allergic response of the subject to the allergen; wherein the allergen antibody inhibitor of Formula I is:

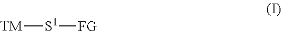

wherein TM is a targeting moiety for an antigen binding site (ABS) wherein the targeting moiety comprises a peanut allergen protein epitope or mimotope peptide sequence, each having a selective electrostatic affinity for the ABS of an allergen reactive antibody, wherein the peanut allergen protein epitope or mimotope peptide sequence comprises a protein epitope or mimotope peptide sequence of an Ara H1 protein, an Ara H2 protein, an Ara H3 protein, or an Ara H6 protein; FG is a reactive functional group capable of forming a site-directed covalent bond to the amine moiety of a lysine amino acid of the allergen reactive antibody; and S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and wherein when the antibody inhibitor binds to the ABS, and the effective concentration of the FG near the lysine amino acid of the allergen reactive antibody increases to irreversibly inhibit the allergen reactive antibody by the site-directed covalent bond formed by the peanut allergen antibody inhibitor.
47. The method of embodiment 46, wherein the FG comprises a conjugate of an isothiocyanate, an isocyanate, an alkyne, a bromine, an acrylamide, or a maleimide.
48. The method of embodiment 46 or 47, wherein the FG is a conjugate of an isothiocyanate or isocyanate.
49. The method of any one of embodiment 46-48, wherein the FG is a conjugate of an isothiocyanate.
50. The method of any one of embodiment 46-49, wherein the FG is isocyanate.
51. The method of any one of embodiment 46-50, wherein S¹ comprises an oligomer of monomers of amino acids comprising y monomers of amino acids.
52. The method of any one of embodiment 46-51, wherein S¹ comprises an oligomer of monomers of saccharides comprising y number of saccharide monomers.
53. The method of any one of embodiment 46-52, wherein y is about 1 to about 15.
54. The method of any one of embodiment 46-53, wherein y is about 3 to about 15.
55. The method of any one of embodiment 46-54, wherein y oligomers is about 5 to about 15.
56. The method of any one of embodiment 46-55, wherein y is about 10 to about 15.
57. The method of any one of embodiment 46-56, wherein y is about 1 to about 5.
58. The method of any one of embodiments 46-57, wherein S¹ comprises an oligomer of ethylene glycol monomers comprising y number of monomers of ethylene glycol, wherein y is about 2 to about 20.
59. The method of any one of embodiments 46-58, wherein y number of monomers of ethylene glycol is about 2 to about 15.
60. The method of any one of embodiments 46-59, wherein y number of monomers of ethylene glycol is about 5 to about 10.
61. The method of any one of embodiments 46-60, wherein y number of monomers of ethylene glycol is about 8.
62. The method of any one of embodiments 46-61, wherein the peanut allergen protein epitope or mimotope peptide sequence comprises any one of SEQ ID NO: 1 to SEQ ID NO: 25.
63. The method of any one of embodiments 46-62, wherein the peanut allergen protein epitope or mimotope peptide sequence comprises any one of SEQ ID NO: 26 to SEQ ID NO: 89.
64. The method of any one of embodiments 46-63, wherein the peanut allergen protein epitope or mimotope peptide sequence comprises any one of (SEQ ID NO: 90), (SEQ ID NO: 91), (SEQ ID NO: 92), and a portion thereof, wherein the portion thereof comprises about 3 to about 15 contiguous amino acids of SEQ ID NO: 90, SEQ ID NO: 91, or SEQ ID NO: 92.
65. The method of any one of embodiments 46-64, wherein the peanut allergen protein epitope or mimotope peptide sequence comprises any one of (SEQ ID NO: 90), (SEQ ID NO: 91), (SEQ ID NO: 92), and a portion thereof, wherein the portion thereof comprises about 5 to about 15 contiguous amino acids of SEQ ID NO: 90, SEQ ID NO: 91, or SEQ ID NO: 92.
66. The method of any one of embodiments 46-65, wherein the peanut allergen protein epitope or mimotope peptide sequence comprises any one of (SEQ ID NO: 90), (SEQ ID NO: 91), (SEQ ID NO: 92), and a portion thereof, wherein the portion thereof comprises about 8 to about 15 contiguous amino acids of SEQ ID NO: 90, SEQ ID NO: 91, or SEQ ID NO: 92.
67. The method of any one of embodiments 46-66, wherein the peanut allergen protein epitope or mimotope peptide sequence comprises any one of (SEQ ID NO: 90), (SEQ ID NO: 91), (SEQ ID NO: 92), and a portion thereof, wherein the portion thereof comprises about 10 to about 15 contiguous amino acids of SEQ ID NO: 90, SEQ ID NO: 91, or SEQ ID NO: 92.
68. The method of any one of embodiments 46-67, wherein the peanut allergen protein epitope or mimotope peptide sequence comprises any one of SEQ ID NO: 5, SEQ ID NO: 93, or SEQ ID NO: 94.
69. The method of any one of embodiments 46-68, wherein the peanut allergen protein epitope or mimotope comprises an Ara H1 allergen protein epitope or mimotope comprising an amino acid sequence of any one of: SEQ ID NO: 26 to 59.
70. The method of any one of embodiments 46-69, wherein peanut allergen protein epitope or mimotope comprises an Ara H2 allergen protein epitope or mimotope comprising an amino acid sequence of any one of SEQ ID NO: 1 to SEQ ID NO: 13.
71. The method of any one of embodiments 46-70, wherein the peanut allergen protein epitope or mimotope comprises an Ara H2 allergen protein epitope or mimotope comprising an amino acid sequence of any one of SEQ ID NO: 1 to13, SEQ ID NO: 60 to 75, and SEQ ID NO: 90 to 92.
72. The method of any one of embodiments 46-71, wherein the peanut allergen protein epitope or mimotope comprises an Ara H3 allergen protein epitope or mimotope comprising an amino acid sequence of any one of: SEQ ID NO: 76 to 89.
73. The method of any one of embodiments 46-72, wherein the peanut allergen protein epitope or mimotope comprises an Ara H6 allergen protein epitope or mimotope comprising an amino acid sequence of any one of SEQ ID NO: 14 to SEQ ID NO: 25.
74. The method of any one of embodiments 46-73, wherein the inhibitor comprises any one of structures cMI-Arah2-A, cMI-Arah2-B, cMI-Arah2-C, cMI-Arah2-D, cMI-Arah2-ABt, or cMI-Arah6-A.
75. The method of any one of embodiments 46-74, wherein the inhibitor is formulated as a composition comprising a pharmaceutically acceptable excipient, carrier, or diluent.
76. The method of any one of embodiments 46-75, wherein inhibition of the peanut allergen is determined using a Rat Basophil Leukemia cells (RBL) degranulation inhibition assay.
77. The method of any one of embodiments 46-76, wherein the route of administration is an intradermal, intramuscular, subcutaneous or intravenous injection.
78. The method of any one of embodiments 46-77, wherein the route of administration is oral or nasal.
79. The method of any one of embodiments 46-78, wherein the inhibitor is formulated for subcutaneous or intravenous injection, transdermal patches, or oral pills.
80. The method of any one of embodiment 46-79, wherein the, an effective amount of the peanut allergen antibody inhibitor may be a dosage of about 0.1 mg/kg to about 100 mg/kg. 81. The method of any one of embodiment 46-80, wherein the, an effective amount of the peanut allergen antibody inhibitor may be a dosage of about 0.1 mg/kg to about 75 mg/kg.
82. The method of any one of embodiment 46-81, wherein the, an effective amount of the peanut allergen antibody inhibitor may be a dosage of about 0.1 mg/kg to about 50 mg/kg. 83. The method of any one of embodiment 46-82, wherein the, an effective amount of the peanut allergen antibody inhibitor may be a dosage of about 0.1 mg/kg to about 25 mg/kg.
84. The inhibitor of any one of embodiments 1-40, wherein the saccharide monomers comprise one or more of glucose, galactose, fructose, xylose, sucrose, lactose, maltose, isomaltulose, trehalose, sorbitol, and mannitol.
85. The inhibitor of any one of embodiments 1-40, wherein the amino acids comprise one or more of lysine, glycine, alanine, and sarcosine.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Peanut Allergen Induced Degranulation

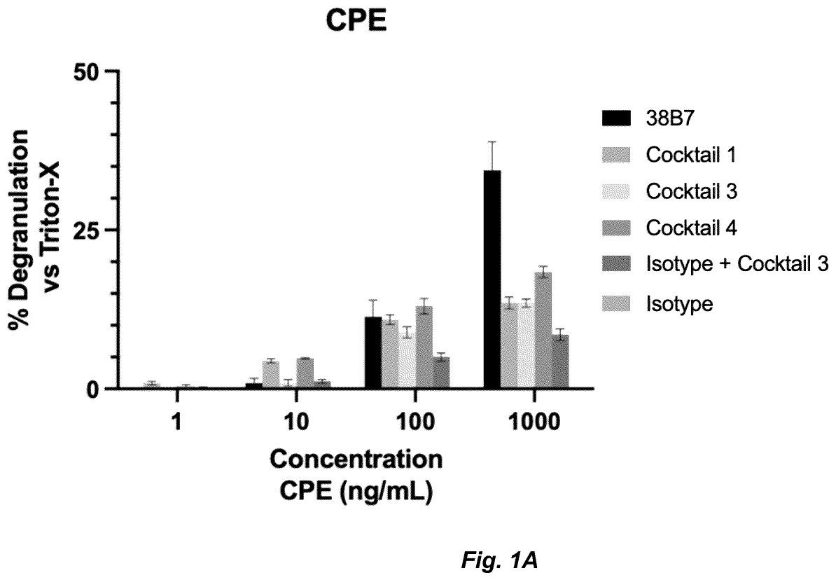

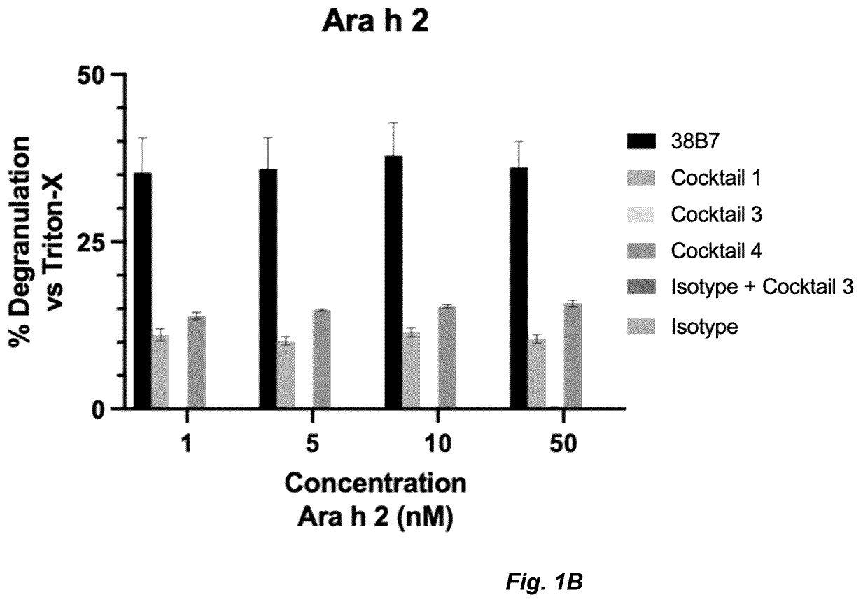

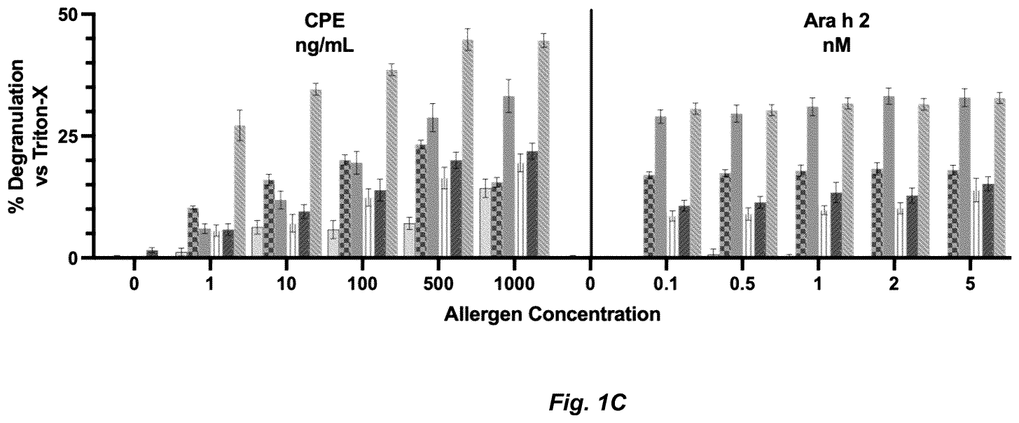

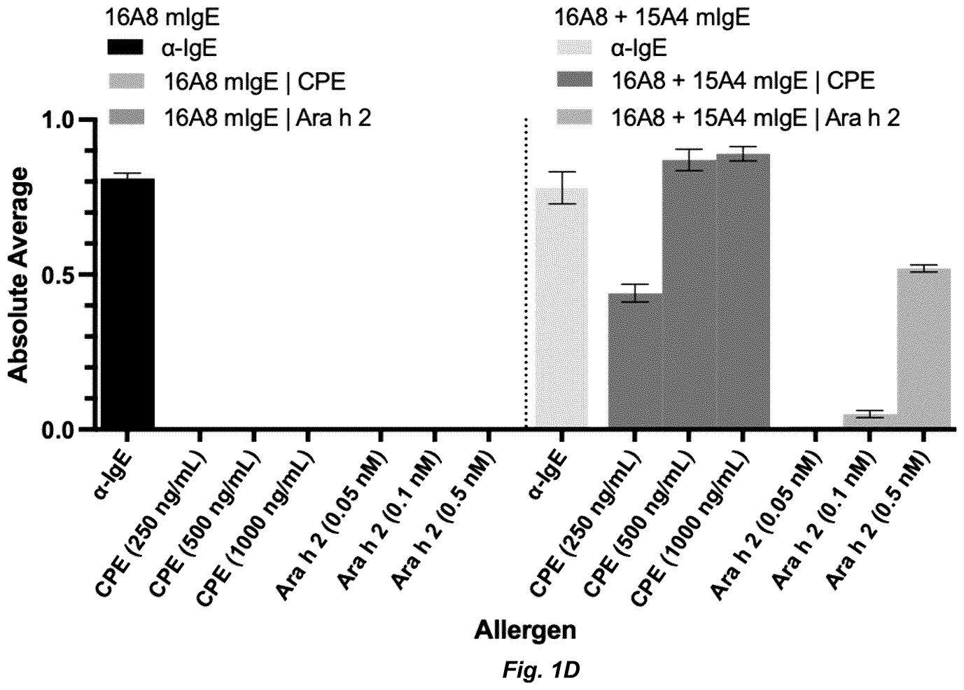

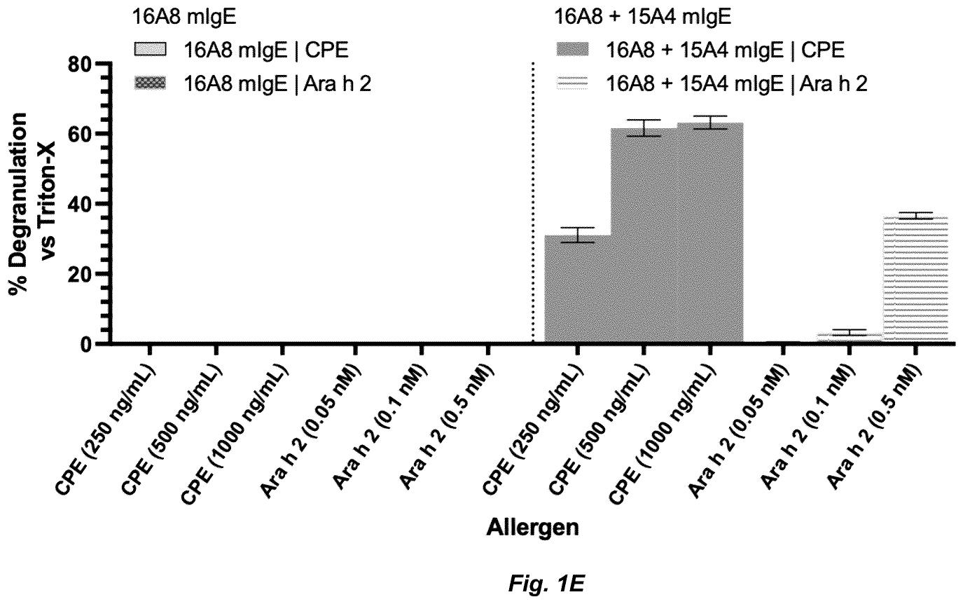

FIGS. 1A-B illustrate crude peanut extract (CPE) and Ara h 2 protein were evaluated for their ability to induce an allergic response from RBL-SX38 cells primed with several mIgE combinations. Priming include 38B7 mIgE alone (Column 1), cocktail 1 (Column 2), cocktail 4 (Column 3), cocktail 3 (Column 4), isotype+cocktail 3 (Column 5), or isotype mIgE alone (Column 6). CPE allergen concentrations of 1, 10, 100, and 1000 ng/ml were evaluated for their ability to elicit degranulation. Ara h 2 allergen concentrations of 1, 5, 10, and 50 nM were evaluated for their ability to elicit degranulation. Data is plotted as the mean of a triplicate measurement +/−SEM.

FIG. 1C illustrates crude peanut extract (CPE) and Ara h 2 protein were evaluated for their ability to induce an allergic response from RBL-SX38 cells primed with several mIgE combinations or peanut allergic patient samples. For each graph, priming includes cocktail 1 (Column 1), cocktail 2 (Column 2), cocktail 3 (Column 3), patient 1 (Column 4), patient 2 (Column 5), or patient 3 (Column 6). CPE allergen concentrations of 0, 1, 10, 100, 500, and 1000 ng/ml were evaluated for their ability to elicit degranulation. Ara h 2 allergen concentrations of 0, 0.1, 0.5, 1, 2, and 5 nM were evaluated for their ability to elicit degranulation. Data is plotted as the mean of a triplicate measurement +/−SEM.

FIGS. 1D-E illustrate crude peanut extract (CPE) and Ara h 2 protein were evaluated for their ability to induce an allergic response from RBL-SX38 cells primed with several mIgE combinations. Priming include 16A8 mIgE alone or 15A4+16A8 mIgE together. CPE allergen concentrations of 250, 500, and 1000 ng/mL were evaluated for their ability to elicit degranulation. Ara h 2 allergen concentrations of 0.05, 0.1, and 0.5 nM were evaluated for their ability to elicit degranulation. Data is plotted as the mean of a triplicate measurement+/−SEM.

### Example 2. Peanut Allergen Induced Inhibition

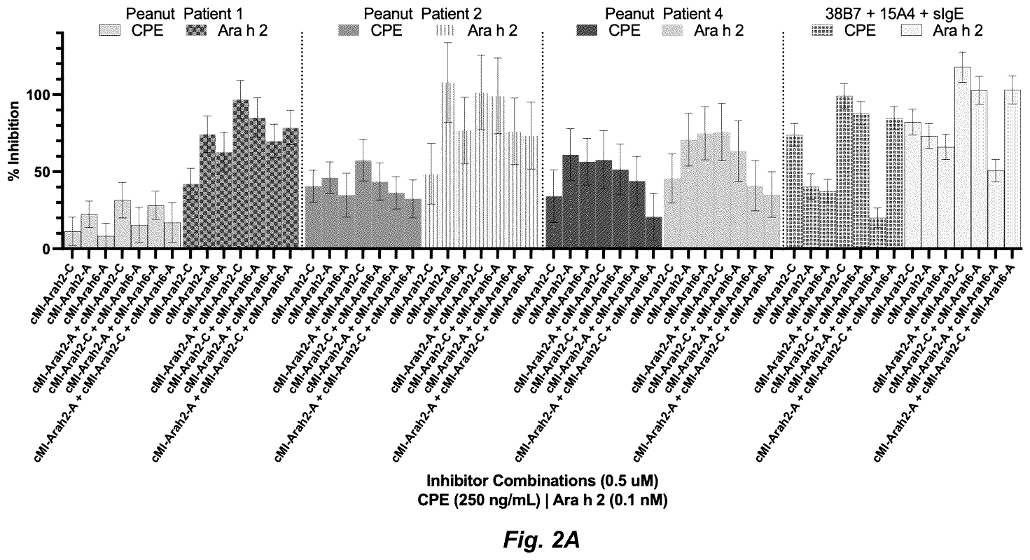

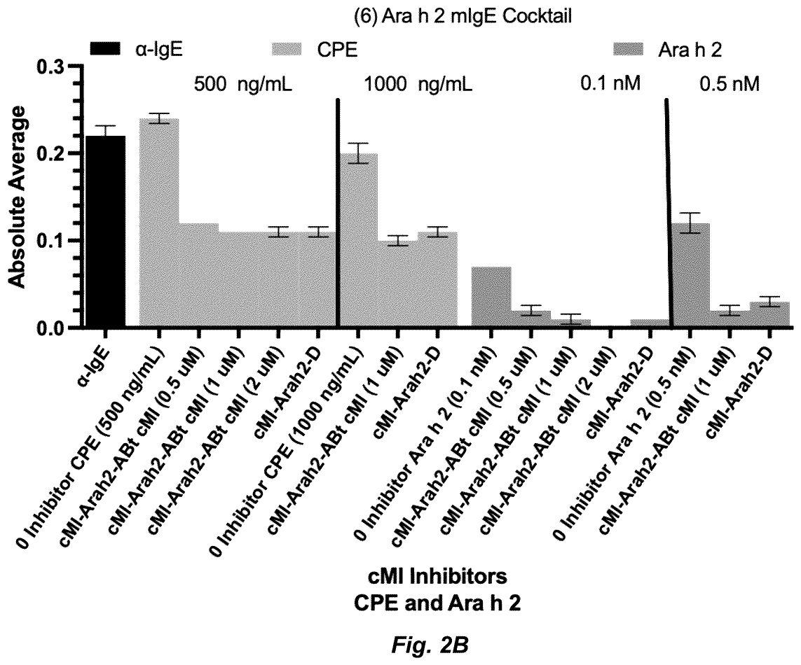

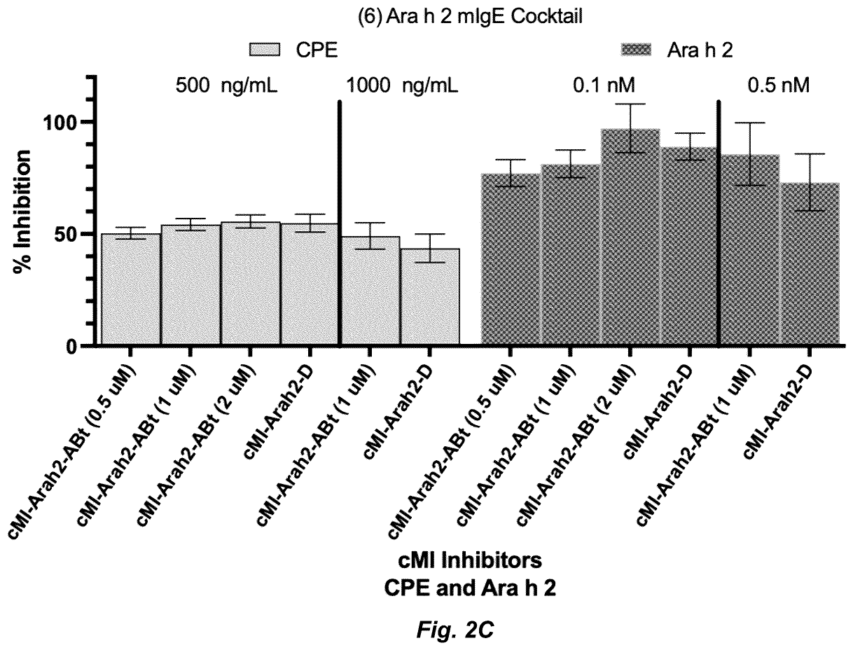

FIG. 2A illustrates covalent monovalent inhibitors (cMIs) were tested alone and in combination with one another to evaluate their ability to inhibit peanut allergen specific degranulation, elicited by crude peanut extract (CPE) or Ara h 2 protein. Arah2-A, Arah2-B, and Arah6-A cMIs were kept at a constant 0.5 uM concentration in well to inhibit allergen specific degranulation whether they were alone or in combination. Cells were challenged with either 250 ng/ml CPE or 0.1 nM Ara h 2 using RBL-SX38 cells primed with peanut allergen specific patient samples or mIgE combinations. Priming included patient 1, patient 2, patient 3, or a cocktail 3. Inhibitors were incubated overnight with primed RBL-SX38 cells, then washed away before allergen challenge. Data is plotted as the mean of a triplicate measurement +/−SEM.

FIGS. 2B-C illustrate covalent monovalent inhibitors (cMIs) were tested to evaluate their ability to inhibit peanut allergen specific degranulation, clicited by crude peanut extract (CPE) or Ara h 2 protein. cMI-Arah2-ABt or cMI-Arah2-D cMIs were compared where cMI-Arah2-ABt cMI was evaluated at 0.5, 1, or 2 uM concentrations in well for their ability to inhibit allergen specific degranulation. Cells were challenged with either 500 ng/ml or 1000 ng/ml CPE or 0.1 or 0.5 nM Ara h 2 using RBL-SX38 cells primed with a peanut allergen specific cocktail 4. Inhibitors were incubated overnight with primed RBL-SX38 cells, then washed away before allergen challenge. Data is plotted as the mean of a triplicate measurement+/−SEM.

### Example 3. Kinetic/Mechanistic Experiments Inhibitor Incubation Time Before Washing

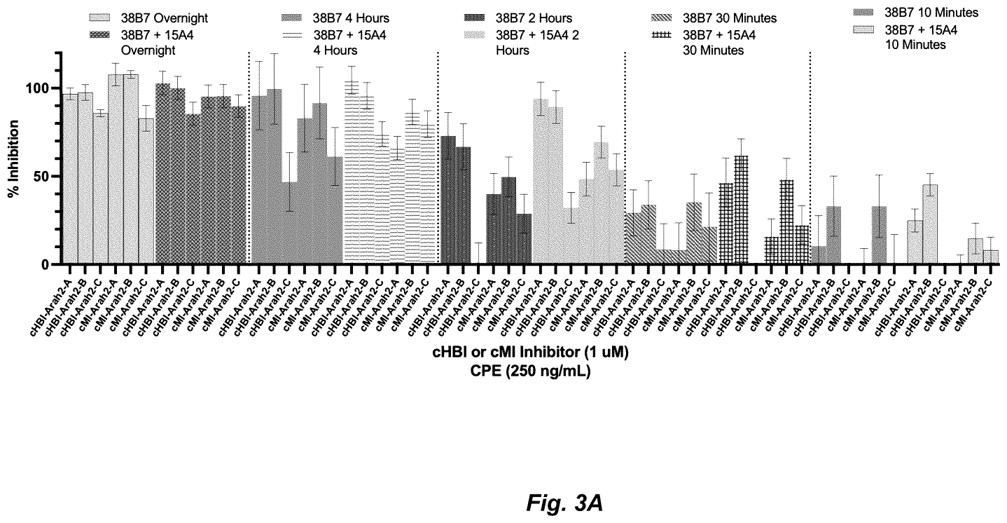

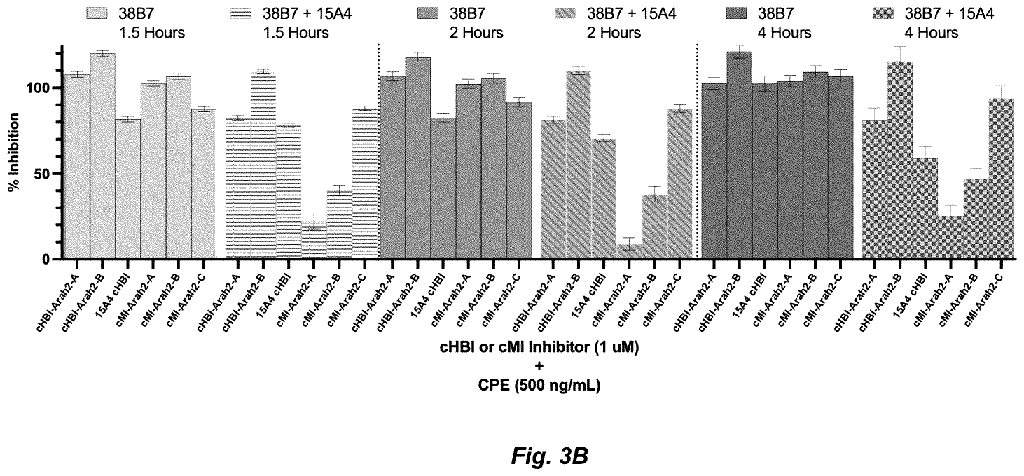

FIG. 3A illustrates covalent monovalent inhibitors (cMIs) and covalent heterobivalent inhibitors (cHBIs) were tested to evaluate their ability to independently inhibit peanut allergen specific degranulation clicited by crude peanut extract (CPE) in a time dependent manner. Arah2-A, Arah2-B, and Arah2-C cMIs or cHBIs were kept at a constant 1.0 uM concentration in well to inhibit allergen specific degranulation. Cells were challenged with 250 ng/ml CPE using RBL-SX38 cells primed with peanut allergen specific mIgE alone or in combination. The priming included 38B7 mIgE alone or 38B7+15A4 mIgE together. Inhibitors were incubated for either 10 minutes, 30 minutes, 2 hours, 4 hours, or overnight with primed RBL-SX38 cells, then washed away before allergen challenge. Data is plotted as the mean of a triplicate measurement +/−SEM.

FIG. 3B illustrates covalent monovalent inhibitors (cMIs) and covalent heterobivalent inhibitors (cHBIs) were tested to evaluate their ability to independently inhibit peanut allergen specific degranulation elicited by crude peanut extract (CPE) in a time dependent manner while in competition with the allergen. Arah2-A, Arah2-B, and Arah2-C cMIs or cHBIs were kept at a constant 1.0 uM concentration in well to inhibit allergen specific degranulation. Cells were simultaneously challenged with 500 ng/ml CPE using RBL-SX38 cells primed with peanut allergen specific mIgE alone or in combination. The priming included 38B7 mIgE alone or 38B7 +15A4 mIgE together. Inhibitors and allergen were incubated together for either 1.5 hours, 2 hours, or 4 hours with primed RBL-SX38 cells. Data is plotted as the mean of a triplicate measurement+/−SEM.

### Example 4. In Vivo Efficacy-Cutaneous and Systemic Anaphylaxis

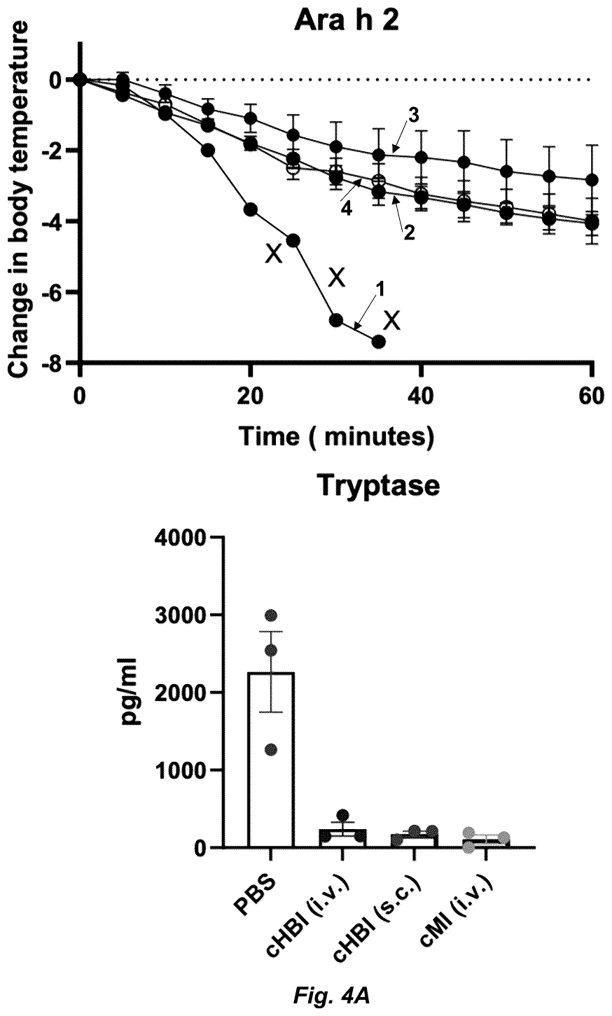

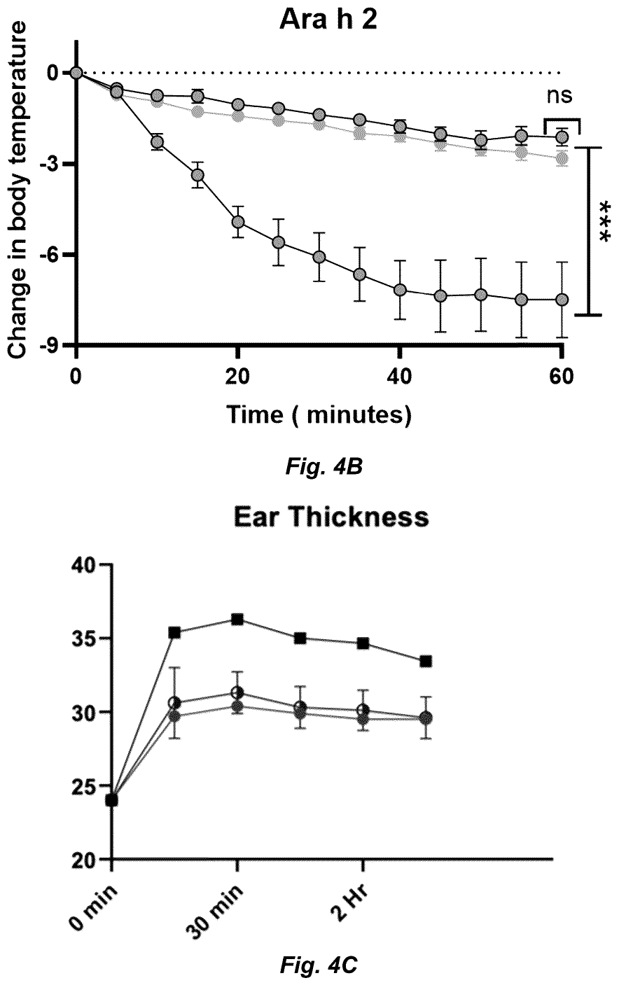

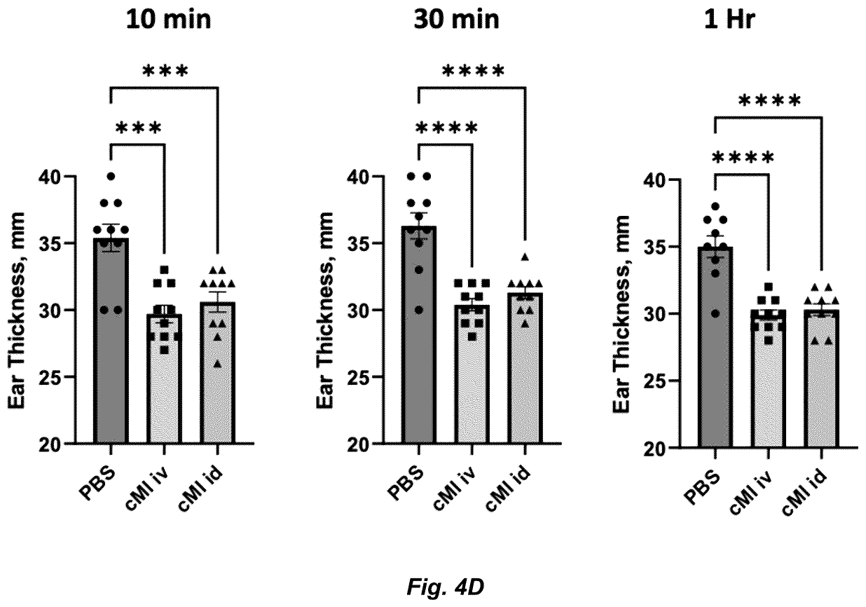

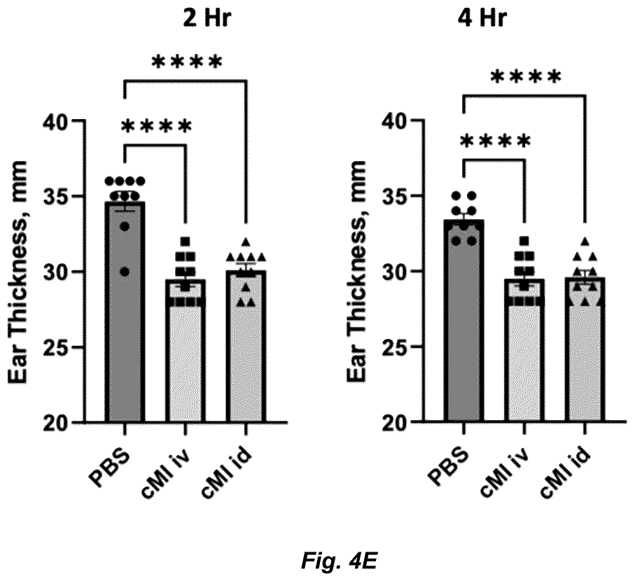

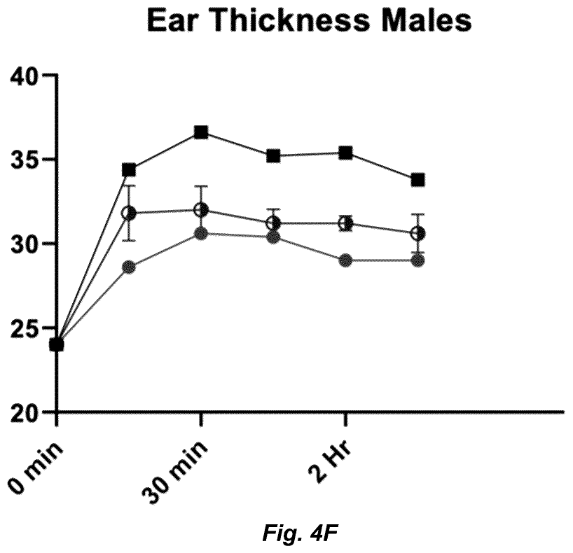

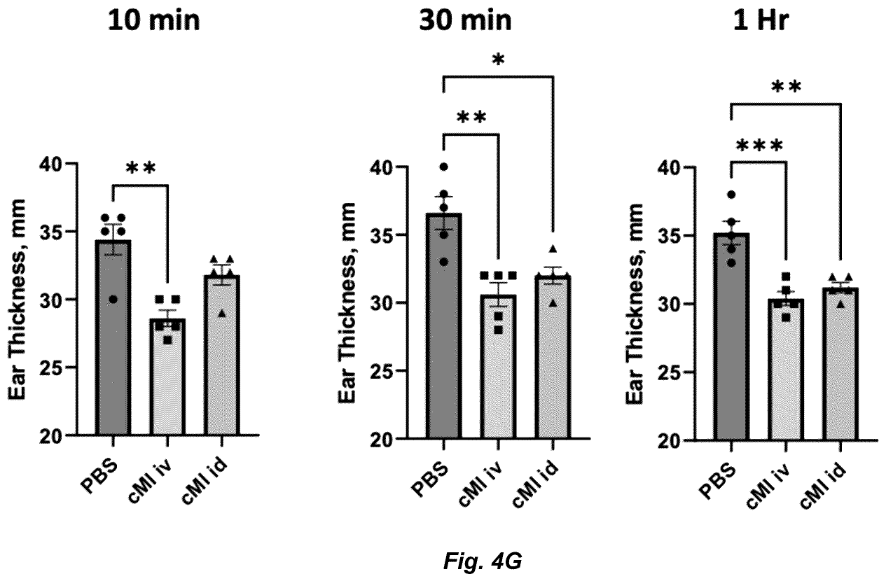

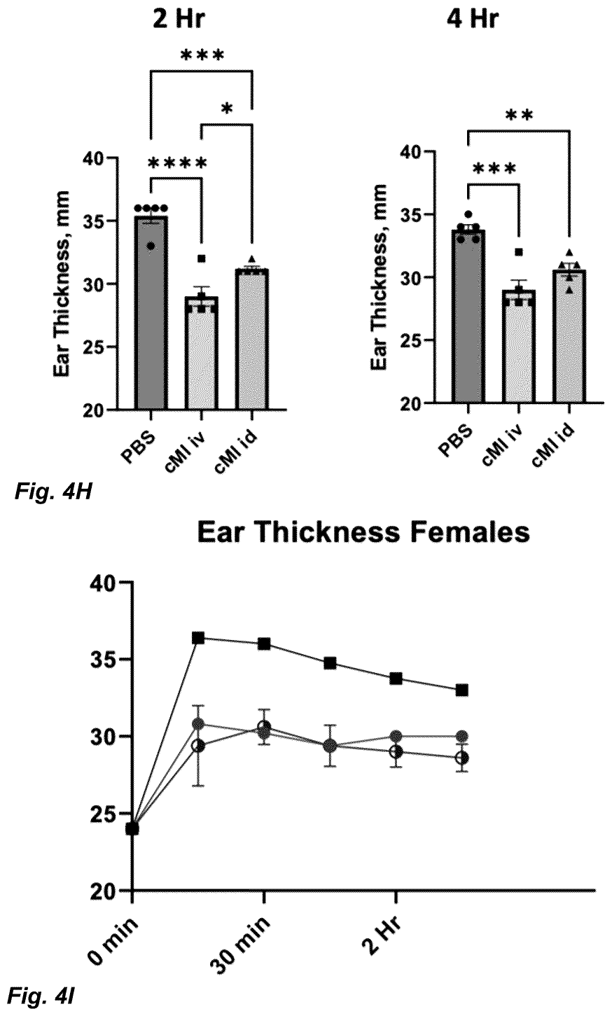

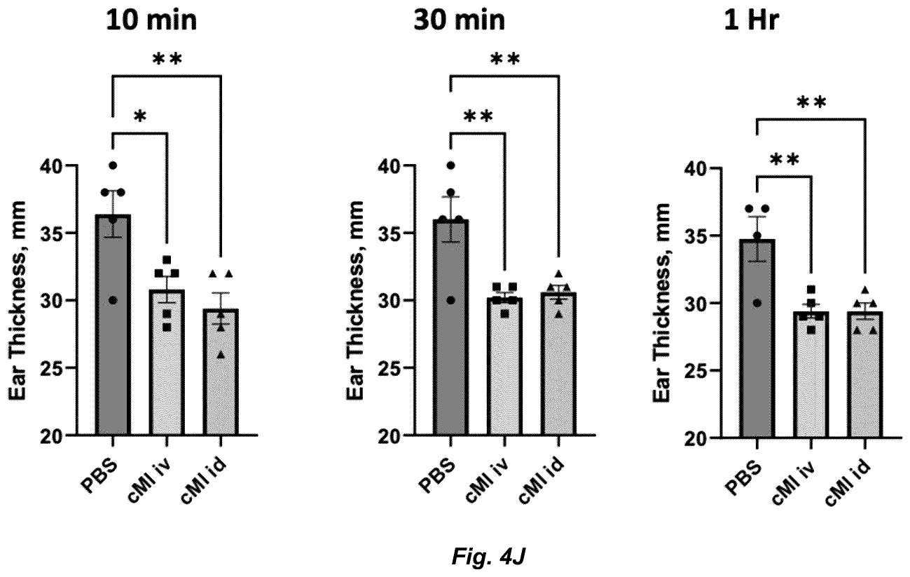

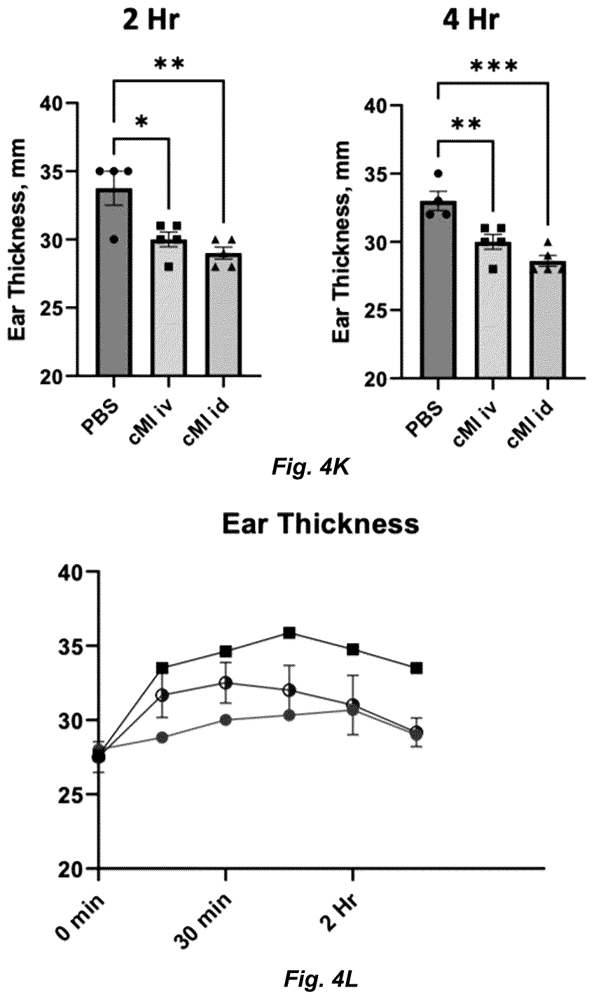

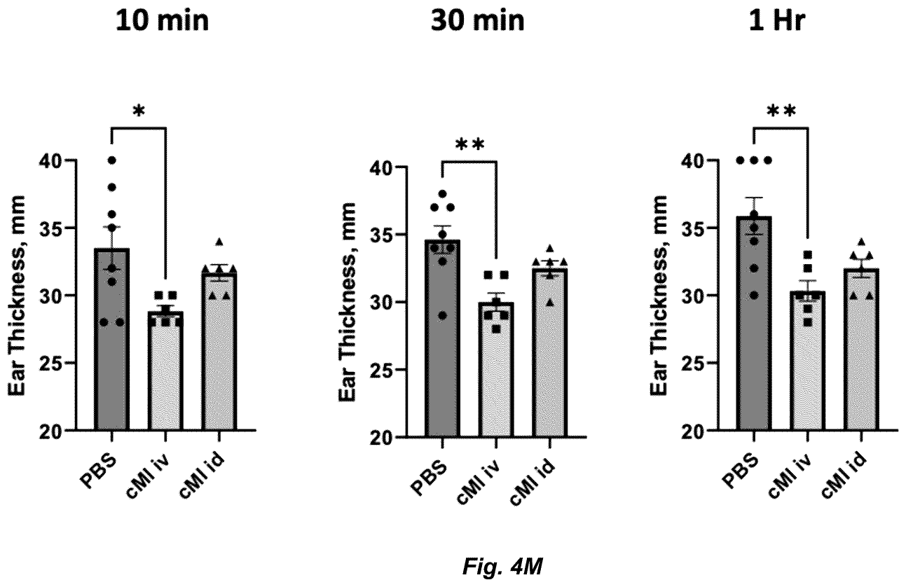

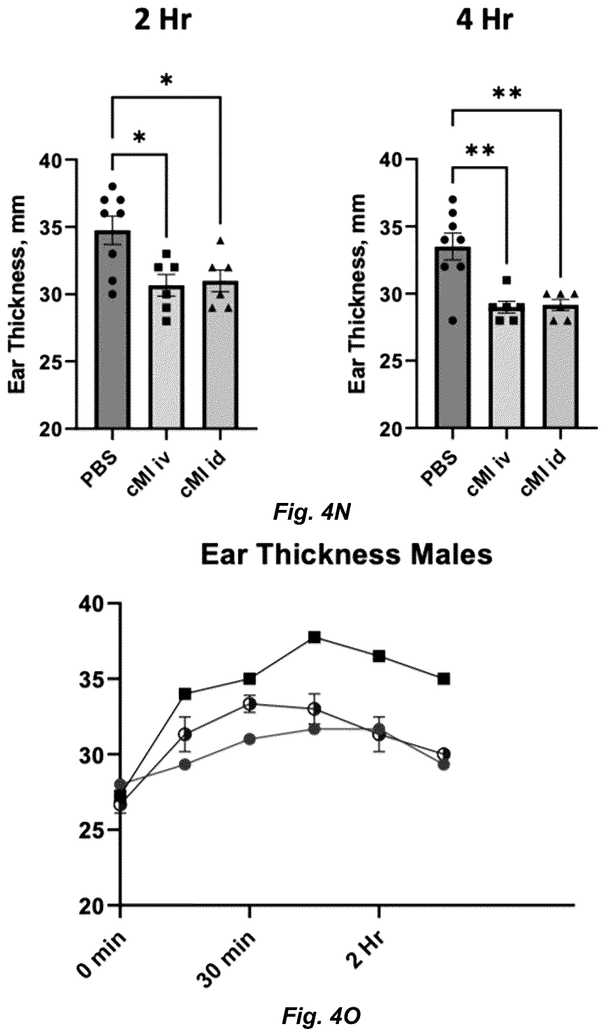

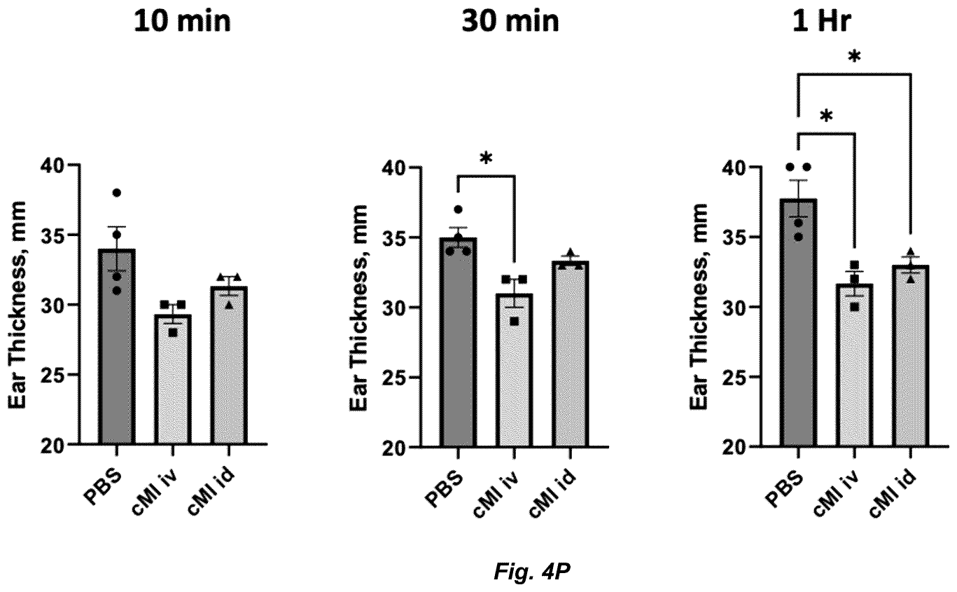

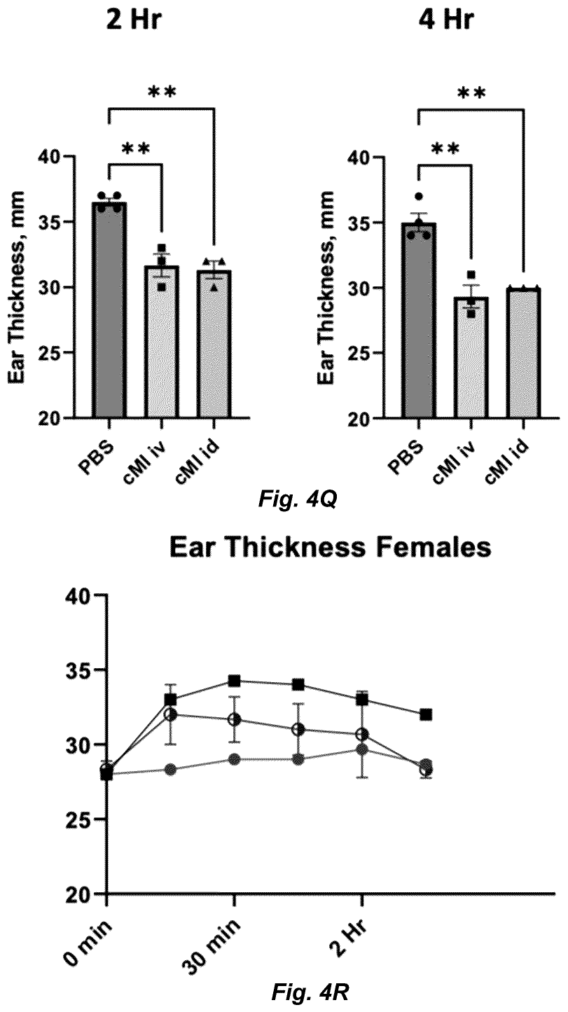

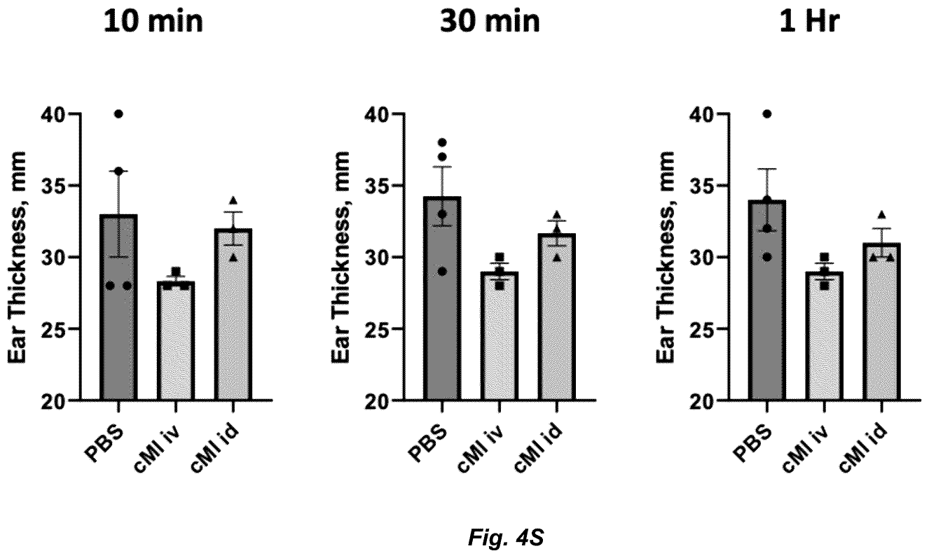

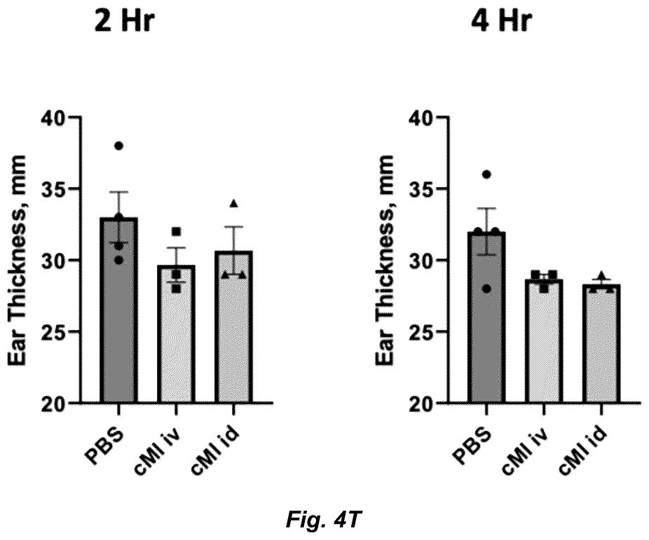

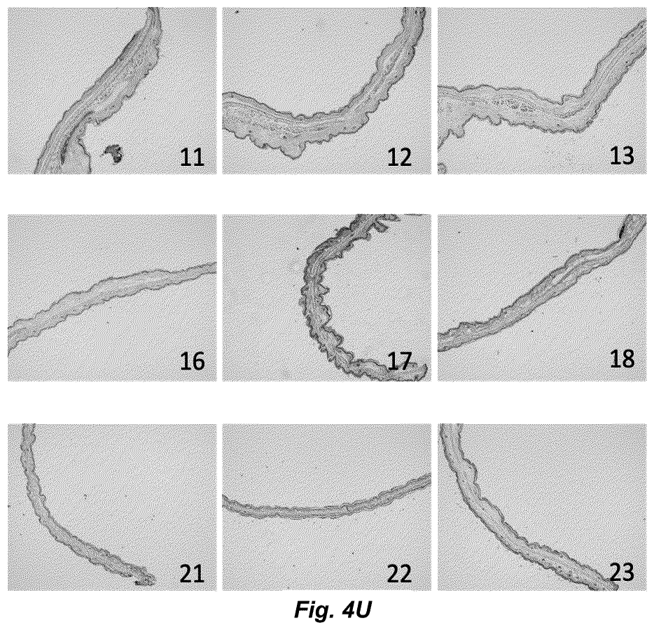

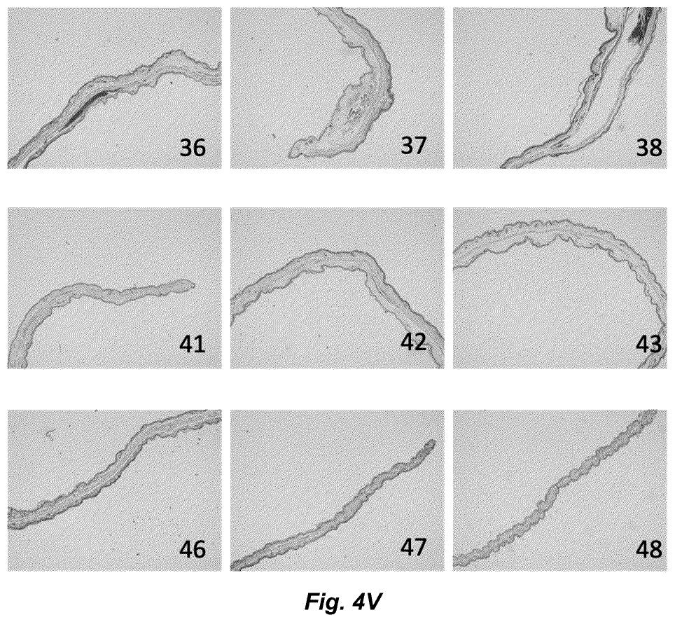

FIG. 4A illustrates covalent monovalent inhibitors (cMIs) and covalent heterobivalent inhibitors (cHBIs) were tested to evaluate their ability to inhibit peanut allergen specific degranulation in HuNSGS mice, clicited by Ara h 2 protein and compared to a PBS control group. Arah2 epitope-B inhibitors (cMI-Arah2-B & cHBI-Arah2-B) were administered through I.V. injection (at 20 nmoles for each inhibitor), while administration of cHBIs were compared using both I.V. and S.C. injections. HuNSGS mice, sensitized with 6 peanut allergen specific mIgE were challenged with 1 ng Ara h 2, 24 hours after peanut allergen inhibitor injections. The antibodies included in sensitization were a combination of 38B7+13D9+8F3+5D7+15A7+16A8 mIgE. Change in body temperature was recorded over the course of 60 minutes after the allergen challenge, where X indicates a mouse died at marked time point. Mouse serum was collected and tryptase levels were evaluated (pg/mL) from each inhibitor treatment group, compared against the PBS controls.

FIG. 4B illustrates covalent monovalent inhibitors (cMIs) and covalent heterobivalent inhibitors (cHBIs) were tested to evaluate their ability to inhibit peanut allergen specific degranulation in HuNSGS mice (n=6), elicited by Ara h 2 protein and compared to a PBS control group. Arah2-B cMIs and cHBIs were administered through S.C. injections. HuNSGS mice, sensitized with 6 peanut allergen specific mIgE were challenged with 1 ng Ara h 2, 24 hours after peanut allergen inhibitor injections. The antibodies included in sensitization using cocktail 1. Change in body temperature was recorded over the course of 60 minutes after the allergen challenge. P-values were determined using one-way ANOVA Tukey's multiple comparison test where ns indicates non-significant.

FIGS. 4C-E illustrate covalent monovalent inhibitors (cMIs) were tested to evaluate their ability to inhibit peanut allergen specific degranulation in HuNSGS mice (n=10), clicited by Ara h 2 protein and compared to a PBS control group. 20 nmol of cMI-Arah2-A was administered through I.V. or I.D. injections to each respective group. HuNSGS mice, sensitized with 1 μg of cocktail 1 were challenged with 10 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male and female mice. P-values were determined using one-way ANOVA where significance was marked when P<0.05 (*), 0.01 (**), 0.001 (***), or 0.0001 (****).

FIGS. 4F-H illustrate covalent monovalent inhibitors (cMIs) were tested to evaluate their ability to inhibit peanut allergen specific degranulation in HuNSGS mice (n=5), clicited by Ara h 2 protein and compared to a PBS control group. 20 nmol of cMI-Arah2-A was administered through I.V. or I.D. injections to each respective group. HuNSGS mice, sensitized with 1 ug of cocktail 1 were challenged with 10 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male mice. P-values were determined using one-way ANOVA where significance was marked when P<0.05 (*), 0.01 (**), 0.001 (***), or 0.0001 (****).

FIGS. 41-K illustrate covalent monovalent inhibitors (cMI-Arah2-A) were tested to evaluate their ability to inhibit peanut allergen specific degranulation in female HuNSGS mice (n=5), elicited by Ara h 2 protein and compared to a PBS control group. 20 nmol of cMI-Arah2-A was administered through I.V. or I.D. injections to each respective group. HuNSGS mice, sensitized with 1 ug of cocktail 1 were challenged with 10 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male and female mice. P-values were determined using one-way ANOVA where significance was marked when P<0.05 (*), 0.01 (**), 0.001 (***), or 0.0001 (****).

FIGS. 4L-N illustrate covalent monovalent inhibitors (cMI-Arah2-A) were tested to evaluate their ability to inhibit peanut allergen specific degranulation in HuNSGS mice (n=10), elicited by Ara h 2 protein and compared to a PBS control group. 20 nmol of cMI-Arah2-A was administered through I.V. or I.D. injections to each respective group. HuNSGS mice, sensitized with 1 μg of cocktail 1 were challenged with 1 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male and female mice. P-values were determined using one-way ANOVA where significance was marked when P<0.05 (*), 0.01 (**), 0.001 (***), or 0.0001 (****).

FIGS. 40-Q illustrate covalent monovalent inhibitors (cMI-Arah2-A) were tested to evaluate their ability to inhibit peanut allergen specific degranulation in male HuNSGS mice (n=5), elicited by Ara h 2 protein and compared to a PBS control group. 20 nmol of cMI-Arah2-A was administered through I.V. or I.D. injections to each respective group. HuNSGS mice, sensitized with 1 μg of cocktail 1 were challenged with 1 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male and female mice. P-values were determined using one-way ANOVA where significance was marked when P <0.05 (*), 0.01 (**), 0.001 (***), or 0.0001 (****).

FIGS. 4R-T illustrate covalent monovalent inhibitors (cMI-Arah2-A) were tested to evaluate their ability to inhibit peanut allergen specific degranulation in female HuNSGS mice (n=5), clicited by Ara h 2 protein and compared to a PBS control group. 20 nmol of cMI-Arah2-A was administered through I.V. or I.D. injections to each respective group. HuNSGS mice, sensitized with 1 ug of cocktail 1 were challenged with 1 ng Ara h 2, 24 hours after I.V. peanut allergen inhibitor injections, or the same day as I.D. injections. Change in car thickness (mm) was recorded at 0, 10, 30, 60, 120, and 240 minutes after the allergen challenge in male and female mice. P-values were determined using one-way ANOVA where significance was marked when P <0.05 (*), 0.01 (**), 0.001 (***), or 0.0001 (****).

FIGS. 4U-V illustrate covalent monovalent inhibitors (cMI-Arah2-A) were tested to evaluate their ability to inhibit peanut allergen specific degranulation in HuNSGS mice (n=10), clicited by Ara h 2 protein and compared to a PBS control group. 20 nmol of cMI-Arah2-A was administered through I.V. or I.D. injections to each respective group. HuNSGS mice, sensitized with 1 μg of cocktail 1 were challenged with 1 ng Ara h 2, 24 hours after I.V. peanut allergen inhibitor injections, or the same day as I.D. injections. At the end of the study, skin histology (H&E staining) was performed. The image and corresponding mouse are labeled.

### Example 5. Degranulation Assays

Rat Basophil Leukemia cells (RBL) degranulation inhibition assay was performed as previously described except for the addition of DNP-specific cHBL inhibitor. RBL-2H3 cells (0.5×10⁶ cells/mL) were cultured on 96-well tissue culture plate overnight. The cells were primed with 25/75% of anti-DNP TgE/anti-cyclinA IgE (1 ug/mL in total antibody concentration). The wells were washed once with dPBS followed by incubation with DNP cHBL inhibitor (1 uM) for 5 hour. DNP-BSA conjugates synthesized as previously described were added into wells with various concentrations (0.01-10,000 ng/mL in Tyrodes buffer) to initiate degranulation. After 90 mM incubation, p-nitrophenyl N-acetyl-β-D-glucosamine solution (1 mM in pH 4.5 citrate buffer) was added to allow for enzymatic cleavage reaction by β-hexosaminidase in cell supernatant solution for 45 min. The reaction was stopped by glycine buffer (1 mM in pH 10.7 solution) and absorbance of product at 405 nm was read to analyze the data. Triton-X (1% (v/v) in DI water) was used as a positive control to normalize the percent degranulation curve (Miyake et al., Cytotechnology. 2024 Apr. 4; 76 (3): 341-349; Dearman et al., Toxicology. Volume 206, Issue 2, 15 January 2005, Pages 195-205; Handlogten et al., Nat. Chem. Biol. 9, 789-795 (2013)).

The degranulation assays followed this basic procedure: (1) RBL cells previously primed with IgEs (either from monoclonal sources or mouse sera from mouse sensitization below) were incubated with cMIs for varying amounts of time, (2) cells were washed to remove any unbound or unconjugated cMIs, (3) allergen was added to stimulate degranulation. Briefly, 50,000 cells were incubated in a 96-well tissue culture plate and either mixtures of monoclonal antibodies to a concentration of 1 μg/mL or dilutions of mouse sera were added for 24 hours. Cells were then washed with sterile PBS and cMI compounds were added at various dilutions for varying time points. Cells were then washed with tyrodes buffer and degranulation was triggered using either dansyl-BSA or penicillin-BSA as previously described (Junutula et al., Nat Biotechnol. 2008; 26 (8): 925-932). Percent inhibition was calculated by dividing percent degranulation with cMI's by control without cMI for same allergen concentration. For some experiments, after incubating with inhibitors for 24 hours, cells were washed and allowed to incubate in cell culture media between 24-72 hours before testing degranulation response.

### Example 6. Pharmaceutical Dosage Forms

The following formulations illustrate representative pharmaceutical dosage forms that may be used for the therapeutic or prophylactic administration of a compound (i., an inhibitor) of a formula described herein, a compound specifically disclosed herein, or a pharmaceutically acceptable salt or solvate thereof, or a composition thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine (pH adjustment to 5-7) | q.s. |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A peanut allergen antibody inhibitor of Formula I: 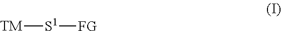
wherein
TM is a targeting moiety for an antigen binding site (ABS) of a peanut allergen reactive antibody wherein the targeting moiety comprises a peanut allergen protein epitope or mimotope peptide sequence, each having a selective electrostatic affinity for the ABS, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
FG is a reactive functional group capable of forming a site-directed covalent bond to a lysine amino acid amine moiety of the allergen reactive antibody; and
S¹ is a variable length spacer comprising oligomers of ethylene glycol, amino acids, saccharides, hydrocarbons, fluorinated hydrocarbons, or a combination thereof, wherein the spacer is conjugated by one or more amide bonds to the TM and the FG.

2. The inhibitor of claim 1, wherein the FG comprises a conjugate of an isothiocyanate, an isocyanate, an alkyne, a bromine, an acrylamide, or a maleimide.

3. The inhibitor of claim 2, wherein the FG is a conjugate of an isothiocyanate or isocyanate.

4. The inhibitor of claim 1, wherein S¹ comprises an oligomer of ethylene glycol monomers comprising y monomers of ethylene glycol, wherein y is about 2 to about 20.

5. The inhibitor of claim 4, wherein y is about 4 to about 10.

6. The inhibitor of claim 1, wherein the Ara H2 or the Ara H6 protein epitope or mimotope comprises any one of:
a.
(SEQ ID NO: 1)
NLRPCEQHLMQKIQRD;
b.
(SEQ ID NO: 2)
ERDPYSPSQDPYSPS;
c.
(SEQ ID NO: 3)
SDRLQGRQQ;
d.
(SEQ ID NO: 4)
RRCQSQLER;
e.
(SEQ ID NO: 5)
HASARQQWEL;
f.
(SEQ ID NO: 6)
RQQEQQFKRELRNLPQQ;
g.
(SEQ ID NO: 7)
PQRCDLE;
h.
(SEQ ID NO: 8)
CDLEVESGGRDRY;
i.
(SEQ ID NO: 9)
CEALQQIMENQSD;
j.
(SEQ ID NO: 10)
CNELNEFENNQR;
k.
(SEQ ID NO: 11)
PRPCEQHLMQKI;
l.
(SEQ ID NO: 12)
ELQGDRRRCQSQLERA;
m.
(SEQ ID NO: 13)
DPYSPSDRRGAGSS;
n.
(SEQ ID NO: 14)
MRRERGRGQDSSSS;
o.
(SEQ ID NO: 15)
KPCEQHIMQRI;
p.
(SEQ ID NO: 16)
YDSYDIR;
q.
(SEQ ID NO: 17)
CDELNEMENTQR;
r.
(SEQ ID NO: 18)
CEALQQIMENQCD;
s.
(SEQ ID NO: 19)
KRELRMLPQQ;
t.
(SEQ ID NO: 20)
CNFRAPQRCDLDV;
u.
(SEQ ID NO: 21)
GEQEQYDSYNFGSTRSSDQ;
v.
(SEQ ID NO: 22)
QDRQ;
w.
(SEQ ID NO: 23)
SSERQVD;
x.
(SEQ ID NO: 24)
IRSTRSSDQQQR;
and
y.
(SEQ ID NO: 25)
QDRQMV.

7. The inhibitor of claim 1, wherein the Ara H1 protein, the Ara H2 protein, or the Ara H3 protein epitope or mimotope comprises any one of SEQ ID NO: 26 to SEQ ID NO: 89 or any one of SEQ ID NO: 5, SEQ ID NO: 13, SEQ ID NO: 23, SEQ ID NO: 93, and SEQ ID NO: 94.

8. The inhibitor of claim 1, wherein the Ara H2 protein epitope or mimotope comprises one or more of:
(SEQ ID NO: 90)
AHASARQQWELQGDRRCQSQLERANLRPCE,
(SEQ ID NO: 91)
EDSYERDPYSPSQDPYSPSPYDRRGAGSS,
(SEQ ID NO: 92)
QFKRELRNLPQQCGLRAPQRCDLEVESGGR,
and a portion thereof, wherein the portion thereof comprises about 3 to about 15 contiguous amino acids of SEQ ID NO: 90, SEQ ID NO: 91, or SEQ ID NO: 92.

9. The inhibitor of claim 1, wherein the inhibitor is:

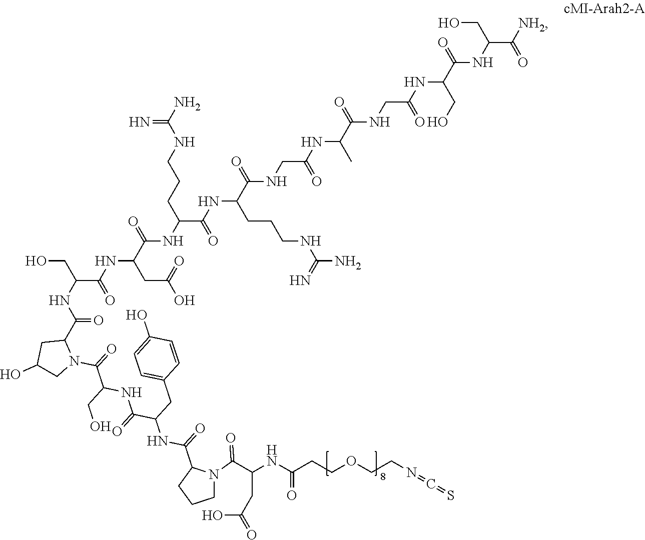

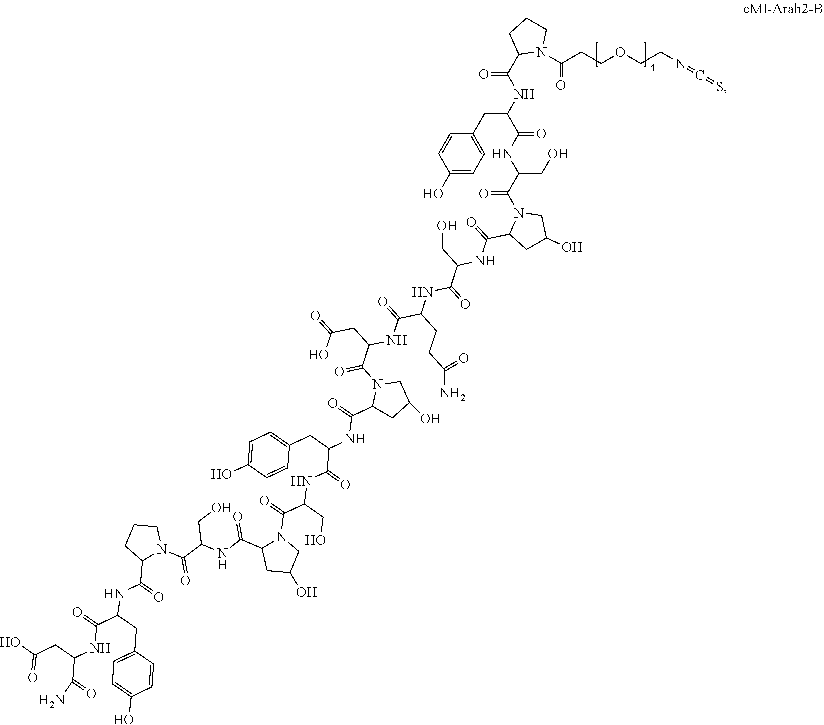

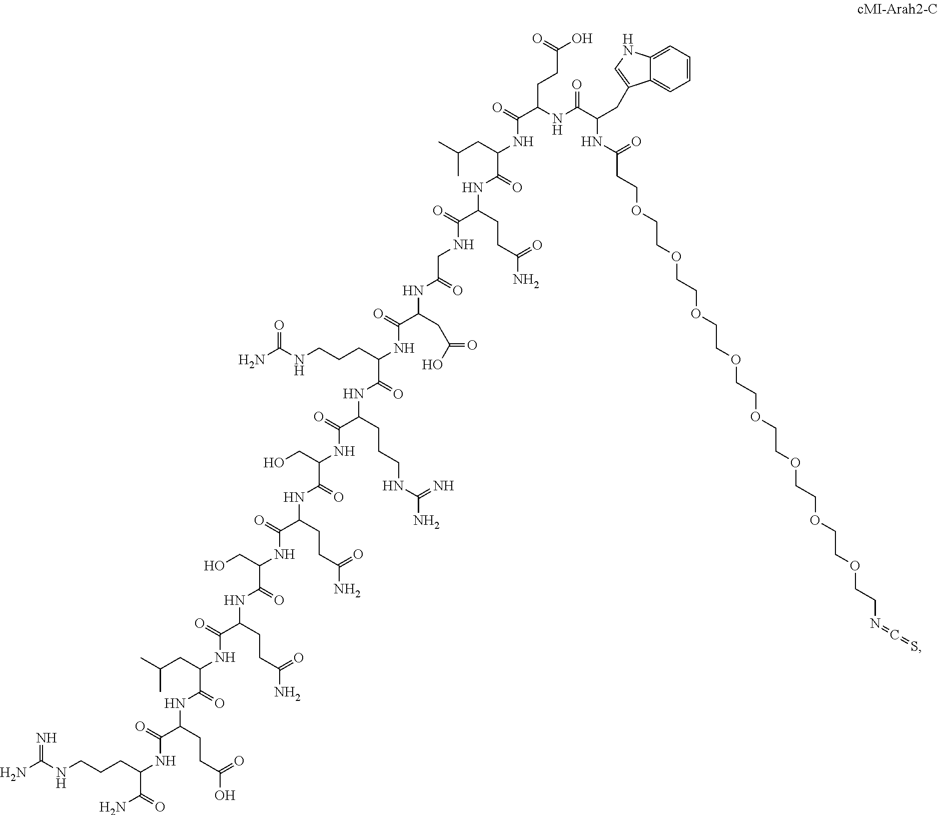 [SMILES: CC(C)CC(NC(=O)C(CCC(=O)O)NC(=O)C(Cc1c[nH]c2ccccc12)NC(=O)CCOCCOCCOCCOCCOCCOCCOCCOCCN=C=S)C(=O)NC(CCC(N)=O)C(=O)NCC(=O)NC(CC(=O)O)C(=O)NC(CCCNC(N)=O)C(=O)NC(CCCNC(=N)N)C(=O)NC(CO)C(=O)NC(CCC(N)=O)C(=O)NC(CO)C(=O)NC(CCC(N)=O)C(=O)NC(CC(C)C)C(=O)NC(CCC(=O)O)C(=O)NC(CCCNC(=N)N)C(N)=O]

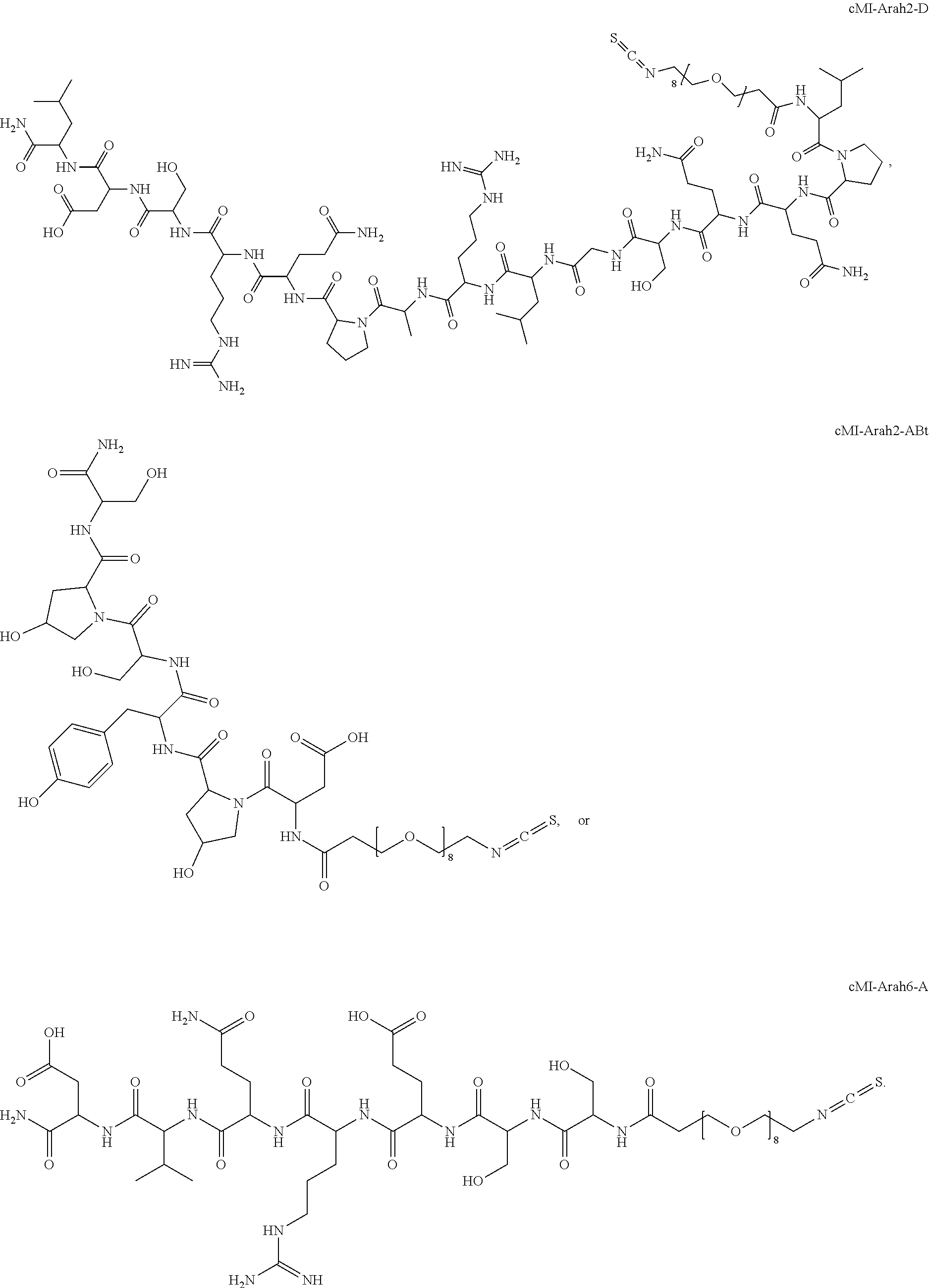

10. A composition comprising the inhibitor of claim 1 and a pharmaceutically acceptable excipient, carrier, or diluent.

11. A method of inhibiting or reducing the severity of an allergic response to a peanut allergy comprising administering an effective amount of a peanut allergen antibody inhibitor according to Formula I to a subject a) prior to exposure of the subject to a peanut allergen, b) after exposure of the subject to a peanut allergen, c) during an allergic response of the subject to a peanut allergen, or d) prior to immunotherapy desensitization of a subject requiring immunotherapy desensitization, wherein inhibition of an immunoglobulin antibody to the peanut allergen by the peanut allergen antibody inhibitor inhibits or reduces degranulation of mast cells and basophils, thereby substantially inhibiting or reducing the allergic response of the subject to the peanut allergen;
wherein the peanut allergen antibody inhibitor of Formula I is: 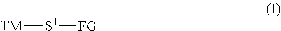
wherein
TM is a targeting moiety for an antigen binding site (ABS) of an peanut allergen reactive antibody wherein the targeting moiety comprises a peanut allergen protein epitope or mimotope peptide sequence, each having a selective electrostatic affinity for the ABS, wherein the peanut allergen protein epitope or mimotope peptide sequence comprises a protein epitope or mimotope peptide sequence of an Ara H1 protein, an Ara H2 protein, an Ara H3 protein, or an Ara H6 protein;
FG is a reactive functional group capable of forming a site-directed covalent bond to the amine moiety of a lysine amino acid of the allergen reactive antibody; and
S¹ is a variable length spacer comprising oligomers of ethylene glycol, amino acids, saccharides, hydrocarbons, fluorinated hydrocarbons, or combination thereof, wherein the spacer is conjugated by one or more amide bonds to the TM and the FG; and
wherein when the antibody inhibitor binds to the ABS, the effective concentration of the FG near the lysine amino acid of the allergen reactive antibody increases to irreversibly inhibit the allergen reactive antibody by the site-directed covalent bond formed by the peanut allergen antibody inhibitor.

12. The method of claim 11, wherein the FG comprises a conjugate of an isothiocyanate, an isocyanate, an alkyne, a bromine, an acrylamide, or a maleimide.

13. The method of claim 12, wherein the FG is a conjugate of an isothiocyanate or isocyanate.

14. The method of claim 11, wherein S¹ comprises an oligomer of ethylene glycol monomers comprising y monomers of ethylene glycol, wherein y is about 2 to about 20.

15. The method of claim 14, wherein y is about 4 to about 10.

16. The method of claim 11, wherein the Ara H2 or the Ara H6 protein epitope or mimotope comprises any one of:
a.
(SEQ ID NO: 1)
NLRPCEQHLMQKIQRD;
b.
(SEQ ID NO: 2)
ERDPYSPSQDPYSPS;
c.
(SEQ ID NO: 3)
SDRLQGRQQ;
d.
(SEQ ID NO: 4)
RRCQSQLER;
e.
(SEQ ID NO: 5)
HASARQQWEL;
f.
(SEQ ID NO: 6)
RQQEQQFKRELRNLPQQ;
g.
(SEQ ID NO: 7)
PQRCDLE;
h.
(SEQ ID NO: 8)
CDLEVESGGRDRY;
i.
(SEQ ID NO: 9)
CEALQQIMENQSD;
j.
(SEQ ID NO: 10)
CNELNEFENNQR;
k.
(SEQ ID NO: 11)
PRPCEQHLMQKI;
l.
(SEQ ID NO: 12)
ELQGDRRRCQSQLERA;
m.
(SEQ ID NO: 13)
DPYSPSDRRGAGSS;
n.
(SEQ ID NO: 14)
MRRERGRGQDSSSS;
o.
(SEQ ID NO: 15)
KPCEQHIMQRI;
p.
(SEQ ID NO: 16)
YDSYDIR;
q.
(SEQ ID NO: 17)
CDELNEMENTQR;
r.
(SEQ ID NO: 18)
CEALQQIMENQCD;
s.
(SEQ ID NO: 19)
KRELRMLPQQ;
t.
(SEQ ID NO: 20)
CNFRAPQRCDLDV;
u.
(SEQ ID NO: 21)
GEQEQYDSYNFGSTRSSDQ;
v.
(SEQ ID NO: 22)
QDRQ;
w.
(SEQ ID NO: 23)
SSERQVD;
x.
(SEQ ID NO: 24)
IRSTRSSDQQQR;
and
y.
(SEQ ID NO: 25)
QDRQMV.

17. The method of claim 11, wherein the Ara H1, the Ara H2 protein, and the Ara H3 protein epitope or mimotope comprises any one of SEQ ID NO: 26 to SEQ ID NO: 89 or any one of SEQ ID NO: 5, SEQ ID NO: 13, SEQ ID NO: 23, SEQ ID NO: 93, and SEQ ID NO: 94.

18. The method of claim 11, wherein the Ara H2 protein epitope or mimotope comprises one or more of:
(SEQ ID NO: 90)
AHASARQQWELQGDRRCQSQLERANLRPCE,
(SEQ ID NO: 91)
EDSYERDPYSPSQDPYSPSPYDRRGAGSS,
(SEQ ID NO: 92)
QFKRELRNLPQQCGLRAPQRCDLEVESGGR,
and a portion thereof, wherein the portion thereof comprises about 3 to about 15 contiguous amino acids.

19. The method of claim 11, wherein the inhibitor is an inhibitor selected from cMI-Arah2-A, cMI-Arah2-B, cMI-Arah2-C, cMI-Arah2-D, cMI-Arah2-ABt, and cMI-Arah6-A, according to claim 9.

20. The method of claim 11, further comprising administering, either concurrently or sequentially, an effective amount of epinephrine with the peanut allergen antibody inhibitor to reduce or inhibit an anaphylaxis response in a subject exposed to the peanut allergen.
